(19) United States
(12) Patent Application Publication
(10) Pub. No.: US 2021/0213153 A1
(43) Pub. Date: Jul. 15, 2021

(54) AROMA VAPOR DISPENSING SYSTEM

(71) Applicant: Francis KWOK, Singapore (SG)

(72) Inventor: Francis KWOK, Singapore (SG)

(21) Appl. No.: 16/967,435

(22) PCT Filed: Jan. 31, 2019
(86) PCT No.: PCT/SG2019/050054

§ 371 Date: Aug. 5, 2020

(30) Foreign Application Priority Data
Feb. 5, 2018 (SG) 10201800964S

(51) Int. Cl. A61L 9/12; A61M 21/00; A61M 21/02

(52) U.S. Cl. CPC A61L 9/125; A61M 21/00; A61L 2209/133; A61M 2021/0016; A61M 2021/0044; A61L 2209/11; A61M 21/02

(57) ABSTRACT

An aroma vapor dispensing system includes an aroma vapor dispenser having multiple concurrently activatable aroma fluid reservoir units, each carrying an aroma fluid vaporizable by an aroma fluid vaporizing element, and each having an identification unit such as a memory associated with a microcontroller storing corresponding a reservoir unit identifier (ID), which can be associated with its aroma fluid. The aroma vapor dispenser communicates with an aroma vapor dispensing management app, which when executed by a personal computing device analyzes the current reservoir unit IDs; and generates an aroma vapor dispensing user interface responsive to user input by which the aroma vapor dispensing management app dynamically converts user-selectable graphical or visual elements into aroma vapor dispensing control signals corresponding to the plurality of current reservoir unit IDs, whereby the aroma vapor dispenser can selectively concurrently activate multiple aroma fluid vaporizing elements provided by the aroma fluid reservoir units on a dynamic basis.

### CROSS-REFERENCE TO RELATED APPLICATION

This application is a national stage application, filed under 35 U.S.C. § 371, of International Patent Application No. PCT/SG2019/050054, which was filed on 31 Jan. 2019, and which is incorporated by reference herein in its entirety.

### TECHNICAL FIELD

Aspects of the present disclosure relate to a system including a set of aroma vapor dispensers that control multiple concurrently activatable aroma fluid reservoir units; and a personal computing device (e.g., a smartphone) having an application program executing thereon configured for (a) communication with the set of aroma vapor dispensers, and (b) providing user interfaces by which relative aroma vapor dispensing outputs among the aroma fluid reservoir units can be dynamically established and communicated to the set of aroma vapor dispensers, thereby enabling the dynamic control of aroma vapor dispensing from the aroma fluid reservoir units, including the dynamic, concurrent dispensing of selectable or variable amounts of aroma vapors from the aroma fluid reservoir units.

### BACKGROUND

The use of essential oils to produce aroma essences or aroma vapors in aromatherapy has in recent times gained recognition as an alternative and complementary therapy to help improve individuals' physical and mental wellbeing. A common technique for aroma essence or aroma vapor application is inhalation, such as by way of an aroma essence or aroma vapor dispensing device. For instance, ultrasonic vaporizer devices have gained popularity for dispensing aroma vapors, as they are safe and easy to operate, and provide dual functionality, namely, aroma vapor dispenser functionality and humidifier functionality. In an ultrasonic vaporizer device, one or more plant essences are incorporated into a water reservoir thereof, and are delivered to the user as an ultrasonically generated aroma vapor composed of a mist of water vapor that carries the plant essences.

A wealth of aromatherapy and essential oil related information can be found in The Complete Book of Essential Oils and Aroma Therapy, 25^{th} Anniversary Edition, by Ann Worwood. Many botanical plant essences, extracted from different parts of the plants, have been the subject of experimental studies, and found to be suitable for treating conditions or illnesses ranging from insomnia, depression, respiratory problems, among others. Increasingly, essential oils are also used in combination with traditional medicines, such as certain antibiotics, to enhance their potency in combating certain illnesses or fighting certain bacteria.

With the increased in studies of morphology and the chemistries of plant essences, and how these essences could benefit users, it is generally agreed that several or many of these plant essences work better or best when different essences, at varying doses, are combined during administration to achieve a synergistic effect, thereby giving rise to enhanced efficacy aromatherapy. In some situations, the same essence mixture may need to vary with respect to the proportions of its constituent essences depending upon user sensitivity or responsiveness, e.g., during administration at different phrases of a treatment program, or even at different times of day, since certain essences may impact or disrupt a natural body rhythm and/or sleep pattern, and hence should not be apply indiscriminately.

Notwithstanding the foregoing, the ability of conventional aroma essence dispensing devices to suitably or appropriately alter the production of aroma vapors during aromatherapy is very limited by virtue of their inherent structural and functional limitations. For example, current conventional or commercially available aroma vapor dispensing devices provide a single aroma fluid reservoir and very limited control functions, and are not suitable for enhanced efficacy aroma therapy administration. A need exists to remedy the deficiencies of conventional aroma vapor dispensing devices.

### SUMMARY

In accordance with an aspect of the present disclosure, an aroma vapor dispensing system includes a set of aroma vapor dispensers providing a plurality of aroma fluid reservoir units, and configured for controlling aroma vapors output from the plurality of aroma fluid reservoir units in accordance with a plurality of aroma vapor dispensing control signals, each aroma fluid reservoir unit configured for carrying an aroma fluid and transforming the aroma fluid into an aroma vapor in accordance with an aroma vapor dispensing control signal, wherein each aroma vapor dispenser includes: a power source; a main control unit electrically coupled to the power source; a communication unit electrically coupled to the main control unit, and configured for signal communication with an electronic device external to the aroma vapor dispenser; a set of aroma fluid reservoir units, each aroma fluid reservoir unit including: a receptacle having a cavity therein for carrying an aroma fluid volume; and an aroma fluid vaporizing device or element fluidically coupled to the cavity, the aroma fluid vaporizing element electrically couplable and activatable by way of the main control unit. The aroma fluid dispenser further includes a set of identification units storing a set of electronically-readable reservoir unit identifiers (IDs) corresponding to the set of aroma fluid reservoir units; and a memory electrically coupled to the main control unit, and configured for storing an aroma vapor dispensing control signal corresponding to each aroma fluid vaporizing element within the set of aroma fluid reservoir units, and which corresponds to an electrical signal level at which the aroma fluid vaporizing element is driven during aroma vapor dispenser operation. The system further includes a personal computing device external to the set of aroma vapor dispensers, where the personal computing device includes: a power supply; a processing unit coupled to the power supply; a set of user interface devices including a display device and a user input device coupled to the processing unit; a communication unit coupled to the processing unit and configured for signal communication with each aroma vapor dispenser within the set of aroma vapor dispensers; a memory coupled to the processing unit, wherein the memory includes an operating system and an aroma vapor dispensing management app, wherein the aroma vapor dispensing management app comprises program instructions which when executed by the processing unit perform aroma vapor dispensing management operations including: establishing signal communication between the personal computing device and the set of aroma vapor dispensers; receiving a plurality of current reservoir unit IDs from the set of aroma vapor dispensers, each reservoir unit ID within the plurality of current reservoir unit IDs associated with a particular aroma fluid reservoir unit within the plurality of aroma fluid reservoir units; automatically generating and presenting on the display device an aroma vapor dispensing user interface that provides visual information content including a plurality of graphical elements, at least some of which are user-selectable, wherein at least one of visual information content colors and graphical element user selectablity depends upon the plurality of current reservoir unit IDs, wherein each user-selectable graphical element interface is correlatable with an electronic signal level at which an aroma fluid vaporizing element of a particular aroma fluid receptacle unit within the plurality of aroma fluid reservoir units can be driven; receiving a user selection corresponding to a first user-selectable graphical element within the aroma vapor dispensing user interface by way of the set of user interface devices; dynamically determining a first plurality of aroma vapor dispensing control signals in response to user selection of the first user-selectable graphical element, each aroma vapor dispensing control signal within the first plurality of aroma vapor dispensing control signals corresponding to a particular aroma fluid receptacle unit within the plurality of aroma fluid receptacle units; and communicating the first plurality of aroma vapor dispensing control signals to the set of aroma vapor dispensers.

The personal computing device can include or be a smart wrist-worn device, a smart head-mounted apparatus, a smartphone, a tablet computing device, a phablet computing device, a laptop computer, and a desktop computer.

In several embodiments, the receptacle of each aroma fluid reservoir unit is removable/replaceable and/or re-fillable; and each aroma fluid reservoir unit further comprises a removable lid or cap configured for re-sealable water-tight engagement with the aroma fluid reservoir unit's receptacle.

For each aroma vapor dispensing unit, each distinct aroma fluid reservoir unit thereof can be configured for carrying its own identification unit. For instance, each aroma fluid dispenser can include a radio frequency identification (RFID) reader coupled to the aroma fluid dispenser's main control unit; and the identification unit of each aroma fluid reservoir unit includes or is an RFID tag storing the reservoir unit ID associated with the aroma fluid reservoir unit, which is communicable to the RFID reader. Alternatively, the identification unit of each aroma fluid reservoir unit includes or is a microcontroller carried by the aroma fluid reservoir unit, which is configured for communicating the reservoir unit ID associated with the aroma fluid reservoir unit to the main control unit.

In several embodiments, for each aroma fluid reservoir unit: the receptacle thereof includes a bottom interior surface, a top interior surface, and at least one side interior surface extending therebetween; the aroma fluid vaporizing element thereof is disposed proximate to the bottom surface of the receptacle; and the bottom surface of the receptacle is configured to reside closer to the surface of the earth than the top surface of the receptacle during activation of the aroma fluid vaporizing element, such that a level of an aroma fluid carried within the aroma fluid reservoir unit's receptacle progressively decreases over time toward the bottom surface of the receptacle.

In various embodiments, each reservoir unit ID within the plurality of current reservoir unit IDs received by the personal computing device is associated with a distinct aroma fluid reservoir unit within the plurality of aroma fluid reservoir units and a particular aroma fluid contained therein that correspondingly produces a particular aroma vapor when vaporized; different user-selectable graphical elements presented by the aroma vapor dispensing user interface correspond to different blend ratios among two or more aroma vapors that are simultaneously dispensable by the plurality of aroma fluid reservoir units; and the first plurality of aroma vapor dispensing control signals corresponds to a specific blend ratio among a first plurality of aroma vapors dispensable by way of concurrent activation of a first plurality of aroma fluid vaporizing elements within the plurality of aroma fluid reservoir units in accordance with the first plurality of aroma vapor dispensing control signals.

Moreover, in various embodiments, each aroma fluid vaporizing element is drivable by way of an aroma vapor dispensing control signal corresponding to an electrical signal level between 0% and 100% of a maximum electrical signal level; and dynamically determining the first plurality of aroma vapor dispensing control signals comprises establishing each aroma fluid vapor dispensing control signal within the first plurality of aroma vapor dispensing control signals on a relative basis with respect to each other aroma fluid vapor dispensing control signal within the first plurality of aroma vapor dispensing control signals, such that a mathematical sum corresponding to the individual fluid vapor dispensing control signal within the first plurality of aroma vapor dispensing control signals represents 100% of the maximum electrical signal level.

The plurality of graphical elements (e.g., including the user-selectable graphical elements) can correspond to a plurality of different colors within portions of the visible optical spectrum. The plurality of graphical elements (e.g., including the user-selectable graphical elements) can be presented as portions of one of (a) a color wheel, (b) a color palette, and (c) color coded geometric shapes.

The aroma vapor dispensing user interface can further include a plurality of visual identifiers presented at predetermined display device coordinates or locations relative to the plurality of user-selectable graphical elements; and the plurality of visual identifiers can include at least one of textual, symbolic, and iconic visual content.

The plurality of visual identifiers can indicate a plurality of intended or expected biological or physiological effects corresponding to exposure of a living mammal to different blend ratios among two or more aroma vapors providable by the plurality of aroma fluid reservoir units when the two or more aroma vapors are concurrently dispensed by the plurality of aroma fluid reservoir units.

The plurality of visual identifiers can include a first identifier associated with a first primary color corresponding to a first subset of graphical elements, a distinct second identifier associated with a second primary color corresponding to a distinct second subset of graphical elements, and a distinct third identifier associated with a third primary color corresponding to a distinct third subset of graphical elements. The plurality of identifiers can also include a fourth identifier corresponding to a gender and associated with a distinct fourth subset of graphical elements. The first, second, third, and fourth visual identifiers can be positioned at different display device coordinates or locations relative to portions of portions of a color spectrum provided by the plurality of graphical elements.

In various embodiments, the aroma vapor dispensing user interface can further provide a graphical representation of user aromatherapy session history data over time.

In various embodiments, each reservoir unit ID within the plurality of current reservoir unit IDs is associated with a particular type of aroma fluid carried by the aroma fluid reservoir unit corresponding to the reservoir unit ID; and the aroma vapor dispensing management app includes program instructions which when executed by the processing unit perform further aroma vapor dispenser management operations including: analyzing the plurality of current reservoir unit IDs; determining whether the aroma fluid reservoir units carry identical or different aroma fluids based on the plurality of current reservoir unit IDs; and at least one of: (a) adjusting the number of user-selectable graphical elements provided by the aroma vapor dispensing user interface if two or more aroma fluid reservoir units carry identical aroma fluids; and (b) establishing a temporally alternating activation condition for the two or more aroma fluid reservoir units carrying identical aroma fluids, in accordance with equal valued aroma vapor dispensing control signals for the two or more aroma fluid reservoir units carrying identical aroma fluids.

The set of aroma vapor dispensers can include a first aroma vapor dispenser that carries a first aroma fluid reservoir unit, and a physically separate second aroma vapor dispenser that carries a second aroma fluid reservoir unit.

The set of aroma vapor dispensers can include a first aroma vapor dispenser configured for simultaneously carrying each of a first aroma fluid reservoir unit and a second aroma fluid reservoir unit. The first aroma vapor dispenser can further include a holder or housing configured for carrying each of the power source, the main control unit, the communication unit, and the memory; the first aroma fluid reservoir unit can carry a first identification unit storing the first electronically-readable ID; the second aroma fluid reservoir unit can carry a second identification unit storing the second electronically-readable ID; and the holder or housing is configured for removable mating engagement of the first and second aroma fluid reservoir units therewith.

In accordance with an aspect of the present disclosure, an aroma vapor dispenser includes: a power source; a main control unit electrically coupled to the power source; a communication unit electrically coupled to the main control unit, and configured for signal communication with an electronic device external to the aroma vapor dispenser; and a set of aroma fluid reservoir units, each aroma fluid reservoir unit having: a receptacle having a cavity therein for carrying an aroma fluid volume; and an aroma fluid vaporizing device or element fluidically coupled to the cavity, the aroma fluid vaporizing element electrically couplable to and activatable by way of the main control unit. The aroma vapor dispenser further includes a set of identification units storing a set of electronically-readable reservoir unit identifiers (IDs) associated with the set of aroma fluid reservoir units, wherein the set of reservoir unit IDs is electromagnetically or electronically communicable to the main control unit; and a memory electrically coupled to the main control unit, and configured for storing an aroma vapor dispensing control signal corresponding to each aroma fluid vaporizing element within the set of aroma fluid reservoir units, and which corresponds to an electrical signal level at which the aroma fluid vaporizing element is driven during aroma vapor dispenser operation.

The aroma vapor dispenser can further includes a housing, platform, or holder; and each aroma fluid reservoir unit within the set of aroma fluid reservoir units can be removably matingly engageable with the housing, platform, or holder.

In accordance with an aspect of the present disclosure, an aroma fluid reservoir unit for an aroma vapor dispenser includes: a receptacle having a cavity therein for carrying an aroma fluid volume; and an aroma fluid vaporizing device or element fluidically coupled to the cavity, the aroma fluid vaporizing element electrically couplable to and activatable by way of the main control unit; and an identification unit storing an electronically-readable reservoir unit identifiers (IDs) corresponding to the fluid reservoir units.

In such an aroma fluid reservoir unit, the electronic identification unit an include or be one of a microcontroller and a radio frequency ID (RFID) tag, which is configured for communicating a reservoir unit ID to an external electronic device or circuit.

### BRIEF DESCRIPTION OF THE DRAWINGS

FIGS. 1A and 1B are schematic illustrations of aroma vapor dispensing systems in accordance with particular non-limiting representative embodiments of the present disclosure.

FIGS. 2A-2B are block diagram showing portions of aroma vapor dispenser electronic element configurations or architectures in accordance with particular non-limiting representative embodiments of the present disclosure.

FIGS. 3A-3I are schematic illustrations of aroma vapor dispensers in accordance with particular non-limiting representative embodiments of the present disclosure, e.g., which can dispense aroma vapors by way of aroma fluid reservoir units that can contain aroma substances such as essential oils diluted in a carrier fluid (e.g., water).

FIG. 4 is a block diagram showing portions of a personal computing device memory contents configuration or architecture in accordance with a particular non-limiting representative embodiment of the present disclosure.

FIGS. 5A-5D are images of user interfaces provided by a set of aroma vapor dispensing user interfaces in accordance with particular non-limiting representative embodiments of the present disclosure.

### DETAILED DESCRIPTION

The FIGS. included herewith show aspects of particular non-limiting representative embodiments in accordance with the present disclosure, and particular structural elements shown in the FIGS. may not be to scale or precisely to scale relative to each other. The depiction of a given element or consideration or use of a particular element number in a particular FIG. or a reference thereto in corresponding descriptive material can encompass the same, an equivalent, or an analogous element or element number identified in another FIG. or descriptive material associated therewith. The presence of “/” in a FIG. or text herein is understood to mean “and/or” unless otherwise indicated. The recitation of a particular numerical value or value range herein is understood to include or be a recitation of an approximate numerical value or value range, for instance, within +/−20%, +/−15%, +/−10%, +/−5%, +/−2.5%, +/−2%, +/−1%, +/−0.5%, or +/−0%. The term “essentially all” can indicate a percentage greater than or equal to 90%, for instance, 92.5%, 95%, 97.5%, 99%, or 100%.

As used herein, the term “set” corresponds to or is defined as a non-empty finite organization of elements that mathematically exhibits a cardinality of at least 1 (i.e., a set as defined herein can correspond to a unit, singlet, or single element set, or a multiple element set), in accordance with known mathematical definitions (for instance, in a manner corresponding to that described in An Introduction to Mathematical Reasoning: Numbers, Sets, and Functions, “Chapter 11: Properties of Finite Sets” (e.g., as indicated on p. 140), by Peter J. Eccles, Cambridge University Press (1998)). Thus, a set includes at least one element (i.e., a set includes 1 or multiple elements). In general, an element of a set can include or be one or more portions of a system, an apparatus, a device, a structure, an object, a process, a physical parameter, or a value (e.g., a signal value or datum value) depending upon the type of set under consideration.

In the present disclosure, signal communication includes or means (a) wireless or wire-based electromagnetic signal, information, and/or data transfer or exchange that occurs directly between systems, apparatuses, devices, or integrated circuitry; or (b) wireless and/or wire-based electromagnetic signal, information, and/or data transfer or exchange that occurs indirectly between systems, apparatuses, devices, or integrated circuitry by way of a communication network that operates by way of electromagnetic signals, such as a personal area network (PAN), a local area network (LAN), a wide area network (WAN), a satellite communication network, the Internet, and/or a cellular or mobile communication network. Network communication includes or means signal, information, and/or data communication by way of one or more of such communication networks.

In the present disclosure, the terms “aromatic or odorous substance,” “aroma substance,” and “aroma composition” can refer to an aromatic, odorous, scented, or fragrant substance or chemical composition, such as an essential oil or perfume. An “aroma fluid” refers to a fluid or liquid that contains, carries, or exists in the form of one or more aromatic or odorous substances, and which can be vaporized. For instance, an aroma fluid can contain one or multiple essential oils (e.g., a blend of multiple essential oils), either diluted with a carrier liquid (e.g., water), or in an undiluted or neat form. An “aroma vapor” refers to a vapor or gas that contains or carries one or more aromatic or odorous substances, e.g., produced by way of vaporization of an aroma fluid.

Structural and Functional Aspects of Representative Aroma Vapor Dispensing Systems

Embodiments in accordance with the present disclosure are directed to systems, apparatuses, devices, circuitry, executable program instruction sets, and user interfaces configured for automatically and dynamically dispensing aroma vapors on a user-selectable basis, including concurrently dispensing multiple aroma vapors in different amounts or quantities relative to each other on a dynamic, near-real time, or essentially real time basis in response to system user input (e.g., visual and/or graphical user input) directed to a user input device (e.g., in association with or during one or more portions of an aroma vapor dispensing session, such as an aromatherapy session).

A system in accordance with an embodiment of the present disclosure includes (a) a set of aroma vapor dispensers having multiple concurrently activatable aroma fluid reservoir units, each of which is configured for carrying an aroma fluid, and dispensing the aroma fluid as an aroma vapor by way of a corresponding aroma fluid vaporizing apparatus, device, or element that is controllable by an aroma vapor dispenser within the set of aroma vapor dispensers; and (b) a personal computing system or device (e.g., a mobile computing device such as a mobile phone/smartphone, a tablet computing device, a phablet computing device, a laptop computer, a smart watch, or smart headgear such as a smart visor or smart glasses) configured for signal communication with the set of aroma vapor dispensers.

In various embodiments, a given aroma vapor dispenser includes a power source (e.g., a battery, which can be rechargeable) and possibly a set of electrical interfaces configured for coupling to a secondary power source (e.g., a powerbank) and/or electrical line power; a main control unit (e.g., a microcontroller; or alternatively, an Application Specific Integrated Circuit (ASIC), or a programmable logic device (PLD) such as a Field Programmable Gate Array (FPGA) having an appropriate configuration bitstream loaded therein, or a state machine); possibly a clock or clock unit, which can be separate from or a form portion of the main control unit; a communication unit (e.g., a Bluetooth™ communication unit); and a memory (e.g., including random access memory (RAM) and read-only memory (ROM), such as electrically erasable programmable ROM (EEPROM)), which in several embodiments can store signals, data, and program instructions executable by the main control unit for performing aroma vapor dispenser control processes. The aroma vapor dispenser control processes include operations directed to establishing electromagnetic or electronic communication with the personal computing device; transferring signals, information, and/or data to and receiving signals, information, and/or data from the personal computing device; and selectively activating/deactivating the aroma fluid vaporizing apparatus, device, or element of each aroma fluid reservoir unit in accordance with aroma vapor dispensing control signals received from the personal computing device, on-demand and/or in association with a schedule (e.g., a programmable schedule) linked with a timer or calendar (e.g., which is maintained in association with operation of the clock unit). The aroma vapor dispenser control processes can also include operations directed to generating or maintaining certain types of aroma therapy session data, such as a time period over which the aroma fluid vaporizing apparatus, device, or element of each aroma fluid reservoir unit was activated during one or multiple recent aroma therapy sessions. Such a time period can include an elapsed aroma therapy session time, and possibly an aroma therapy session identifier (ID) and/or a date stamp.

Each aroma vapor dispenser also typically includes a housing, platform, or holder with which one or multiple aroma fluid reservoir units are selectively engageable (e.g., removably matingly engageable), depending upon the structural configuration of the aroma vapor dispenser under consideration. For instance, a particular aroma vapor dispenser can be configured for carrying a single aroma fluid reservoir unit, or multiple aroma fluid reservoir units, such as between 1-12 aroma fluid reservoir units (e.g., 1, 2, 3, 3, 5, 6, 7, 8, or 10 aroma fluid reservoir units), depending upon embodiment details. In multiple embodiments, a system user can selectively engage different aroma fluid reservoir units with a given aroma vapor dispenser. In some embodiments in which an aroma vapor dispenser is configured for simultaneous engagement with multiple aroma fluid reservoir units, the system user can selectively or flexibly engage a particular aroma fluid reservoir unit with the aroma vapor dispenser at different aroma fluid reservoir unit receiving locations or positions provided by the aroma vapor dispenser's housing, platform, or holder.

In various embodiments, an aroma fluid reservoir unit includes: a receptacle having a cavity therein for carrying a volume of an aroma fluid; and an aroma fluid vaporizing apparatus, device, or element fluidically couplable or coupled to the cavity, and which is electrically couplable to the power source and main control unit of an aroma vapor dispenser within the set of aroma vapor dispensers, and which is activatable or drivable by way of such aroma vapor dispenser's main control unit for vaporizing the aroma fluid within the cavity. Depending upon embodiment details, the aroma fluid vaporizing apparatus, device, or element can include or be a piezoelectric transducer, or a nebulizer (e.g., an atomizing apparatus or atomizer, which can atomize a neat essential oil or essential oil blend without requiring the essential oil or essential oil blend to be in a carrier fluid) in a manner understood by individuals having ordinary skill in the relevant art), or another type of electronic device capable of vaporizing the aroma fluid contained within the cavity.

The aroma fluid reservoir unit further includes an identification unit that stores an electronically-readable or computer-readable ID associated with or corresponding to the aroma fluid reservoir unit and/or its aroma fluid vaporizing apparatus, device, or element, which can be defined as a reservoir unit ID. The reservoir unit ID is communicable to the main control unit of an aroma vapor dispenser with which the aroma fluid reservoir is engaged. Depending upon embodiment details, the reservoir unit ID of a given aroma fluid reservoir unit can be stored in a memory corresponding to a microcontroller carried by a portion of the aroma fluid reservoir unit, or a radio frequency ID (RFID) tag carried by a portion of the aroma fluid reservoir unit, such that the reservoir unit ID can be communicated to the main control unit of an aroma vapor dispenser within the set of aroma vapor dispensers. For instance, in embodiments in which an aroma fluid reservoir unit stores its reservoir unit ID by way of a microcontroller carried by a portion of the aroma fluid reservoir unit, the aroma fluid reservoir unit and the aroma vapor dispenser with which the aroma fluid reservoir is engagable/engaged include counterpart electrical couplings or connectors, such as male and female electrical connector structures (e.g., a pair of pins and a counterpart pair of sockets) by which the aroma fluid reservoir's microcontroller is couplable/coupled to the aroma vapor dispenser's main control unit, in a manner individuals having ordinary skill in the relevant art will readily comprehend. In embodiments in which an aroma reservoir unit stores its reservoir unit ID in an RFID tag, the aroma vapor dispenser includes an RFID reader providing an antenna by which the reservoir unit ID can be read from the RFID tag, in a manner also understood by individuals having ordinary skill in the relevant art.

In various embodiments, the personal computing system or device includes a power supply (e.g., a rechargeable battery), a set of electronic interfaces and associated circuitry for coupling to a secondary power source, and a set of electronic interfaces and associated circuitry for coupling to line power; at least one processing unit (e.g., a microprocessor); a set of user input devices (e.g., a touch screen display, or other input/output devices such as a pen/stylus, or a touchpad, and a display device); a communication unit (e.g., a Bluetooth™ communication unit, and possibly a cellular network communication unit and/or a network communication unit such as a WiFi communication unit); and a memory in which an operating system and program instruction sets reside. The personal computing system can also include one or more removable data storage devices, in a manner readily understood by individuals having ordinary skill in the relevant art.

The memory can store an operating system, and program instruction sets executable by the processing unit(s), including one or more program instruction sets that form portions of or which can be defined as an aroma vapor dispensing management application program or app. In association with its execution, the aroma vapor dispensing management app is configured for performing aroma vapor dispensing management processes or operations including: (a) establishing communication with the set of aroma vapor dispensers; (b) possibly receiving aroma therapy session data corresponding to one or multiple recently completed aroma therapy sessions from the set of aroma vapor dispensing devices; (c) receiving a current set of reservoir unit IDs from the set of aroma vapor dispensing devices, which indicates the particular aroma fluid reservoir units and/or aroma fluid vaporizing apparatuses, devices, or elements thereof that the aroma vapor dispensing device(s) can presently activate for outputting aroma vapors; (d) providing or generating and presenting a set of aroma vapor dispensing user interfaces based on or corresponding to the current set of reservoir unit IDs; (e) dynamically determining, establishing, generating, or retrieving a current set of aroma vapor dispensing control codes and/or signals corresponding to the current set of reservoir unit IDs based upon user input/user selection(s) directed to such user interface(s); and (f) communicating the current set of aroma vapor dispensing control codes and/or signals to the set of aroma vapor dispensers, such that aroma vapor dispensing from the current set of aroma fluid reservoir units can occur or is established in accordance with the current set of aroma vapor dispensing control codes and/or signals.

Each aroma vapor dispensing control code and/or signal is correlated with, corresponds to, or defines an activation or drive signal level (e.g., an electromagnetic or electronic signal level, such as an electrical voltage and/or current level) for a particular aroma fluid vaporizing apparatus, device, or element carried by a particular aroma fluid reservoir unit associated with or corresponding to the current set of reservoir unit IDs. Thus, an aroma vapor dispensing control code and/or signal directed to the aroma fluid vaporizing apparatus, device, or element of a particular aroma fluid reservoir unit is correlated with, corresponds to, or defines an electromagnetic or electronic signal level (e.g., amplitude or magnitude) at which the aroma fluid vaporizing apparatus, device, or element is to be driven for purpose of vaporizing an aroma fluid carried by this aroma fluid reservoir unit. Prior to communicating the current set of aroma vapor dispensing control codes and/or signals to the set of aroma vapor dispensers, the aroma vapor dispensing management app can associate, link, or tag each aroma vapor dispensing control code and/or signal within the current set of aroma vapor dispensing control codes and/or signals with a specific reservoir unit ID within the current set of reservoir unit IDs. Moreover, in some embodiments, the aroma vapor dispensing management app can also associate an activation time interval with some or each of the aroma vapor dispensing control codes and/or signals in the current set of aroma vapor dispensing control codes and/or signals, e.g., to indicate a default or customized aroma therapy session duration across which the current set of aroma fluid dispensing units are to be activated in accordance with such codes and/or signals.

Further to the foregoing, in various embodiments, within the current set of aroma vapor dispensing control codes and/or signals, each aroma vapor dispensing control code and/or signal corresponds to or establishes or defines an activation or drive signal level for the aroma fluid vaporizing apparatus, device, or element of a specific aroma fluid reservoir unit relative to the activation or drive signal level(s) of the other aroma fluid vaporizing apparatus(es), device(s), or element(s) associated with or corresponding to the current set of reservoir unit IDs.

For instance, if (a) the current set of aroma fluid reservoirs provides a pair of aroma fluid reservoir units definable as (i) a first aroma fluid reservoir unit containing a first aroma fluid, and having a first reservoir unit ID (e.g., “1”) and a first aroma fluid vaporizing element that can be driven by way of a first aroma vapor dispensing control signal between 0% and 100% of a maximum drive signal level (e.g., between 0 V and 5 V, inclusive), and (ii) a distinct second aroma fluid reservoir unit containing a second aroma fluid and having a second reservoir unit ID (e.g., “2”) and a distinct second aroma fluid vaporizing element that can be driven by way of a second aroma vapor dispensing control signal between 0% and 100% of the same maximum drive signal level; and (b) the aroma vapor dispensing management app determines that the most-recently received user input directed to the aroma vapor dispensing user interface(s) corresponds to the first aroma vapor dispensing control signal establishing a drive signal level for the first aroma fluid vaporizing element of j % of the maximum drive signal level, the aroma vapor dispensing management app can automatically determine that the second aroma vapor dispensing control signal establishes a relative drive signal level for the second aroma fluid vaporizing element given by k=(100−j) % of the maximum drive signal level, such that j and k are established or defined relative to each other, and (j+k) totals to 100% of the maximum drive signal level. The values of j and k depend upon the specific user input received by way of the user interface(s). In various embodiments, user input that identifies or selects a specific visual or graphical element or a specific set thereof presented by the aroma vapor dispensing user interface(s) determines the values of j and k relative to each other. In some embodiments, the aroma vapor dispensing management app determines current values of j and k in response to user input by way of a lookup table (e.g., directed to a portion of a lookup table that resides in the personal computing device's memory). In other embodiments, the aroma vapor dispensing management app can determine current values of j and k in response to user input by way of calculations corresponding to one or more mathematical algorithms or formulas.

Further with respect to the above representative example, in several embodiments the aroma vapor dispensing management app can communicate the current set of aroma vapor dispensing control signals to the set of aroma vapor dispensers by communicating each of the first reservoir unit ID linked with the value of j and/or a code corresponding thereto, and the second reservoir unit ID linked with the value of k and/or a code corresponding thereto, to the aroma vapor dispenser(s). If neither j nor k equals 0% or 100%, the aroma vapor dispenser(s) can subsequently concurrently activate the first and second aroma fluid vaporizing elements of the first and second aroma fluid reservoir units, respectively, to provide, generate, or output a composite aroma vapor, e.g., an aroma vapor blend, formed by way of vaporization of the first and second aroma fluids based on the determined values of j and k, respectively. In other words, the composite aroma vapor is formed as approximately j% of a first aroma vapor and approximately k% of a second aroma vapor, each of which is respectively formed from vaporization of the first aroma fluid and the second aroma fluid in accordance with the values of j and k. If j equals 100%, the aroma vapor dispenser(s) can simply output a first aroma vapor corresponding to vaporization of the first aroma fluid at the maximum drive signal level, in the absence of vaporization of the second aroma fluid; and if k equals 100%, the aroma vapor dispenser(s) can simply output a second aroma vapor corresponding to vaporization of the second aroma fluid at the maximum drive signal level, in the absence of vaporization of the first aroma fluid.

In a manner analogous to that described above, if (a) the current set of aroma fluid reservoirs provides three aroma fluid reservoir units definable as (i) a first aroma fluid reservoir unit having a first reservoir unit ID (e.g., “1”) and a first aroma fluid vaporizing element that can be driven by way of a first aroma vapor dispensing control signal between 0% and 100% of a maximum drive signal level (e.g., between 0 V and 5 V, inclusive), (ii) a distinct second aroma fluid reservoir unit having a second unit reservoir unit ID (e.g., “2”) and a distinct second aroma fluid vaporizing element that can be driven by way of a second aroma vapor dispensing control signal between 0% and 100% of the maximum drive signal level, and (iii) a third aroma fluid reservoir unit having a third reservoir unit ID (e.g., “3”) and a distinct third aroma fluid vaporizing element that can be driven by way of a third aroma vapor dispensing control signal between 0% and 100% of the maximum drive signal level; and (b) the aroma vapor dispensing management app determines that the most-recently received user input directed to the aroma vapor dispensing user interface(s) corresponds to (i) the first aroma vapor dispensing control signal establishing a drive signal level for the first aroma fluid vaporizing element of p % of the maximum drive signal level, and (ii) the second aroma vapor dispensing control signal establishing a relative drive signal level for the second aroma fluid vaporizing element of q % of the maximum drive signal level, such that (p+q) is less than or equal to 100%, then the aroma vapor dispensing management app can automatically determine that the third aroma vapor dispensing control signal establishes a drive signal level for the third aroma fluid vaporizing element of r=1−(p+q) of the maximum drive signal level, such that p, q, and r are established relative to each other, in accordance with the condition that (p+q+r) totals to 100%. The values of p, q, and r depend upon the specific user input received by way of such user interface(s). In various embodiments, user input that identifies or selects a specific visual or graphical element presented by the aroma vapor dispensing user interface(s) determined the values of p, q, and r relative to each other. In several embodiments, the aroma vapor dispensing management app can communicate the current set of aroma vapor dispensing control signals to the set of aroma vapor dispensers by way of communicating each of the first reservoir unit ID linked with the value of p and/or a code corresponding thereto, the second reservoir unit ID linked with the value of q and/or a code corresponding thereto, and the third reservoir unit ID linked with the value of r and/or a code corresponding thereto to the aroma vapor dispenser(s). In some embodiments, the aroma vapor dispensing management app determines current values of p, q, and r in response to user input by way of a lookup table operation (e.g., directed to a portion of lookup table that resides in the personal computing device's memory). In other embodiments, the aroma vapor dispensing management app can determine current values of p, q, and r in response to user input by way of calculations corresponding to one or more mathematical algorithms or formulas.

Further with respect to the preceding representative example, in several embodiments the aroma vapor dispensing management app can communicate the current set of aroma vapor dispensing control signals to the set of aroma vapor dispensers by communicating each of the first reservoir unit ID linked with the value of p and/or a code corresponding thereto, the second reservoir unit ID linked with the value of q and/or a code corresponding thereto, and the third reservoir unit ID linked with the value of r and/or a code corresponding thereto to the aroma vapor dispenser(s). The set of aroma vapor dispensers can subsequently concurrently activate the first and second aroma fluid vaporizing elements of the first and second aroma fluid reservoir units, respectively, to provide, generate, or output a composite aroma vapor, e.g., an aroma vapor blend, formed by way of vaporization of the first, second, and third aroma fluids based on the determined values of p, q, and r, respectively. In other words, the composite aroma vapor is formed as approximately p % of a first aroma vapor, approximately q % of a second aroma vapor, and approximately r % of a third aroma vapor, each of which is respectively formed from vaporization of the first aroma fluid, the second aroma fluid, and the third aroma fluid in accordance with the values of p, q, and r. In a manner similar to that described above, if any of p, q, or r equals 100%, the aroma vapor dispenser(s) can simply output a first, a second, or a third aroma vapor corresponding to vaporization of the first, second, or third aroma fluid, respectively, in the absence of vaporization of the other aroma fluids.

Similar or analogous considerations to those described above apply to embodiments in which the current set of aroma fluid reservoir units provides four or more aroma concurrently activatable fluid reservoir units.

In view of the above, in various embodiments the values or levels of the individual aroma vapor dispensing control signals within the current set of aroma vapor dispensing control signals, as determined in accordance with user input directed to the set of aroma vapor dispensing user interfaces, are interdependent relative to each other and the maximum drive signal level at which individual aroma fluid vaporizing apparatuses, devices, or elements can be driven.

A given reservoir unit ID can be associated or linked with a predetermined type of aroma fluid, such as a pre-manufactured essential oil blend carried by a particular aroma fluid reservoir unit having the reservoir unit ID under consideration, e.g., which is available for sale individually or as part of a kit. The reservoir unit ID can be pre-programmed into the identification unit of this aroma fluid reservoir unit, in a manner readily understood by individuals having ordinary skill in the relevant art. Additionally or alternatively, a specific reservoir unit ID associated with or corresponding to a replaceable or removable and re-fillable aroma fluid reservoir unit can be associated or linked with a user-customizable or customized type of aroma fluid, such as a user-preferred essential oil or essential oil blend, such as by way of user input directed to the set of aroma vapor dispensing user interfaces. This customized linkage can be stored in the personal computing device's memory.

In some embodiments, the aroma vapor dispensing management app can analyze the current set of reservoir unit IDs to determine whether two or more aroma fluid reservoir units within the current set of aroma fluid reservoir units contain an identical or identical type (e.g., categorical type) of aroma fluid. If so, the aroma vapor dispensing management app can establish or define the current set of aroma vapor dispensing control signals (e.g., in association with determining values of j and k or p, q, and r in a manner described above) such that only one of the two or more aroma fluid reservoir units within the current set of aroma fluid reservoir units that contain an identical or identical type of aroma fluid is activated at any given time, e.g., such that these aroma fluid reservoir units are activated in an alternating manner in accordance with a particular or predetermined alternation time period, e.g., in order to reduce power consumption/save battery life. For instance, the aroma vapor dispensing management app can associate an alternation flag and/or alternation time interval with the current set of aroma fluid dispensing control signals corresponding to these aroma fluid reservoir units, which can be communicated to the set of aroma vapor dispensers.

The information content (e.g., visual information content) provided by the set of aroma vapor dispensing user interfaces and/or the particular user selections available within the set of aroma vapor dispensing user interfaces at a given time can depend or vary based on the particular reservoir unit IDs present within the current set of reservoir unit IDs received from the set of aroma vapor dispensers. Hence, the information content provided by the set of aroma vapor dispensing user interfaces, and/or portions of the set of aroma vapor dispensing user interfaces that are selectable by the user, can differ or change depending upon the particular aroma fluid reservoir unit(s) that are currently activatable by the set of aroma vapor dispensers, and their expected aroma fluid contents. Thus, because each aroma fluid reservoir unit is expected to carry a particular aroma fluid or particular types of aroma fluid in accordance with its reservoir unit ID, the information content provided by the set of aroma vapor dispensing user interfaces, and/or portions of the set of aroma vapor dispensing user interfaces that are selectable by the user, can depend upon the specific aroma fluids carried or expected to be carried by the current set of aroma fluid reservoir units. In various embodiments, the aroma vapor dispensing management app can adaptively generate, retrieve, and/or adjust the particular information content presented by the set of aroma vapor dispensing user interfaces and/or the aroma vapor/aroma vapor blend selections available for user selection depending upon the particular aroma fluid(s) or particular types of aroma fluid(s) that the current set of aroma fluid reservoir units is expected to carry in accordance with the current set of reservoir unit IDs.

Depending upon embodiment details, the information content provided or presented by the set of aroma vapor dispensing user interfaces at any given time includes graphical objects, which can include visual or graphical elements (hereafter “graphical elements” for ease of understanding), user interface controls, and/or textual, iconic, or symbolic visual identifiers, labels, or tags, which can be associated with particular sets or subsets of graphical elements. Particular displayed graphical elements or sets thereof can be user-selectable or designated as user-selectable, whereas other displayed graphical elements or sets thereof can be non-user-selectable or designated as non-user-selectable under the control of the aroma vapor dispensing management app, e.g., in accordance with the current set of reservoir unit IDs.

In some embodiments, at least some graphical elements are displayed in a color coded manner, e.g., which corresponds to or represents portions of a color wheel or color palette, and thus particular graphical elements or subsets thereof are displayed in accordance with a visual color scheme corresponding to portions a standardized visual spectrum, and/or the visible optical spectrum. Thus, each graphical element, or particular sets thereof, can correspond to a particular standardized digital code (e.g., a hexadecimal, octal, binary, or decimal code) that represents a particular color, e.g., which is displayed within the color wheel or color palette in accordance with a standardized visual spectrum. The aroma vapor dispensing management app can map or convert a user selection of a given user-selectable graphical element or set of graphical elements corresponding to a particular color code to a particular set of aroma fluid vaporizing element control signals corresponding to the current set of reservoir unit IDs.

For instance, in Red-Green-Blue (RGB) color space, hexadecimal or hex color code “#397ee5” (or equivalently, decimal color code (57, 126, 229)) corresponds to a specific shade of bright blue, composed of 22.4% red, 49.4% green, and 89.8% blue. The aroma vapor dispensing management app can map or convert this color code to a particular set of aroma fluid vaporizing element control signals. As a representative example provided to aid understanding, for an aroma vapor dispenser providing first, second, and third concurrently activatable aroma fluid reservoir units, each having an aroma fluid vaporizing element that can be driven up to an identical maximum drive signal level (e.g., corresponding to a maximum electrical drive signal amplitude for the aroma fluid vaporizing element), and having corresponding reservoir unit IDs of “A”, “B”, and “C,” based on these reservoir unit IDs the aroma vapor dispensing management app can map or convert user selection of a graphical element corresponding to hex color code #397ee5 to relative aroma fluid vaporizing element control signal values (p, q, r) analogous to those described above as (8%, 12%, 80%), indicating that the aroma fluid vaporizing element of the aroma fluid dispensing unit corresponding to reservoir unit ID “A” is to be driven at 8% of the maximum drive signal level; the aroma fluid vaporizing element of the aroma fluid dispensing unit corresponding to reservoir unit ID “B” is to be driven at 12% of the maximum drive signal level, and the aroma fluid vaporizing element of the aroma fluid dispensing unit corresponding to reservoir unit ID “C” is to be driven at 80% of the maximum drive signal level, where once again, 8%+12%+80% totals to 100% of the maximum drive signal level. In view of the foregoing, the aroma vapor dispensing management app can map or convert a user selection of a given user-selectable graphical element or set of graphical elements corresponding to a particular color/color code to user selection of a particular aroma vapor or composite aroma vapor producible by way of vaporization of the aroma fluids carried by the current set of aroma fluid reservoir units, or equivalently, the aroma fluids corresponding to the current set of reservoir unit IDs.

In an analogous manner, the aroma fluid dispensing management app can map or convert other user selectable graphical elements or sets thereof, e.g., corresponding to other specific color codes, to other relative aroma vaporization control signal combinations in accordance with a current or most-recently received set of reservoir unit IDs, as individuals having ordinary skill in the art will readily understand from the description provided herein. More particularly, for any given user selectable graphical element or set thereof corresponding to or forming a portion of a color wheel or color palette, the aroma fluid dispensing management app can map or convert a color code corresponding to the displayed color of such user selectable graphical element(s) to a plurality of interdependent aroma fluid dispensing control signals in accordance with the current or most-recently received set of reservoir unit IDs. Different mappings between color codes corresponding to user-selectable graphical elements and sets of interdependent aroma fluid dispensing control signals can be pre-determined for different combinations of reservoir unit IDs, and stored as lookup tables in the personal computing device's memory, in a manner individuals having ordinary skill in the relevant art will also readily comprehend.

Visual identifiers can be presented or displayed relative to (e.g., above, below, around, adjacent, or within) particular portions of the color wheel or color palette, such that particular visual identifiers are visually associatable or associated or linked with particular colors. For instance, particular visual identifiers can be presented or displayed proximate or adjacent to predetermined regions of the color wheel or color palette corresponding to primary colors; and/or certain visual identifiers can be presented or displayed proximate or adjacent to predetermined regions of the color wheel or color palette corresponding to specific blend ratios of primary colors.

In several embodiments, each visual identifier indicates an expected or main expected biological or physiological effect associated with the vaporization of one or more aroma fluids carried by the current set of aroma fluid reservoir units, e.g., a mental, emotional, immunomodulatory, aphrodisiac, or other type of effect. For instance, a set of visual identifiers can indicate “mental focus/alertness/clarity,” “anti-cough,” “anti-influenza,” “anti-headache,” “anti-stress,” “sleep,” “sensual,” “female” (e.g., for female aphrodisiac effects), or “male” (e.g., for male aphrodisiac effects). The particular user-selectable graphical elements (e.g., which graphical elements are currently user-selectable and which graphical elements are not currently user-selectable), the particular visual color scheme, and/or the visual identifiers presented by the set of aroma vapor dispensing user interfaces, e.g., corresponding to the color wheel or color palette, can depend upon the current set of reservoir unit IDs that the personal computing device received from the set of aroma vapor dispensers, and hence upon the particular aroma fluids carried or expected to be carried by the corresponding aroma fluid reservoirs.

Relative to the foregoing, the set of aroma vapor dispensing user interfaces presents or displays multiple user-selectable graphical elements of different colors and possibly visual identifiers corresponding thereto, and the aroma vapor dispensing management app can map, convert, or effectively transform a given user selection of a particular graphical element or set thereof to a particular aroma vapor or composite aroma vapor, e.g., a currently selected aroma vapor or composite aroma vapor. The currently selected aroma vapor or composite aroma vapor is detectable by way of the system user's olfactory system during an aromatherapy session, and which can discern, distinguish, or differentiate from other aroma vapors or composite aroma vapors corresponding to user selection of other graphical elements. Thus, a system in accordance with several embodiments of the present disclosure can visually associate or link specific colors corresponding to user-selectable graphical elements with the production of corresponding specific aroma vapors or composite aroma vapors. Moreover, a system in accordance with multiple embodiments of the present disclosure can visually associate or link specific colors corresponding to user-selectable graphical elements, and specific expected biological or physiological effects corresponding to visual identifiers, with the production of corresponding specific aroma vapors or composite aroma vapors. System users can therefore develop and reinforce neural associations between specific information content presented by the set of aroma vapor dispensing user interfaces, e.g., colors and possibly visual identifiers, and specific aroma vapors or composite aroma vapors, and biological or physiological effects associated therewith.

Additionally or alternatively, in certain embodiments the set of aroma vapor dispensing user interfaces can provide or present at least some graphical objects corresponding to or in the form of predetermined geometric shapes (e.g., portions of an ellipse/circle or ring; or a triangle, a square, or other regular or irregular geometric shape having multiple sides), which contain user-selectable visual or graphical objects therein. The aroma vapor dispensing management app can associate different predetermined geometric shapes with different aroma fluid reservoir units in accordance with the current set of dispensing unit IDs. The aroma vapor dispensing management app can control the set of aroma vapor dispensing user interfaces such that visual identifiers presented near each geometric shape indicate the relative percentage of aroma fluid carried by the aroma fluid reservoir unit associated with the geometric shape that will be dispensed in accordance with most-recently received user input. In situations in which two or more aroma fluid reservoir units contain an identical or identical type of aroma fluid, instead of presenting two or more geometric shapes corresponding to these aroma fluid reservoir units and the aroma fluids carried thereby, the aroma vapor dispensing management app can control the aroma vapor dispensing user interface(s) to present a single geometric shape corresponding to this identical or identical type of aroma fluid, and can further establish corresponding aroma vapor dispensing control signals such that these aroma fluid reservoir units are activated in an alternating manner by way of identically valued aroma vapor dispensing control signals such as described above.

In addition to the foregoing, in various embodiments the aroma vapor dispensing management app maintains user history data corresponding a system user, where such user history data includes a number of aromatherapy sessions that the system has initiated or performed for the user (e.g., as a sequential session count, which can be linked with particular calendar dates), and a length or duration (e.g., in minutes) for each aromatherapy session. At least some of the user history data can include or be based on aromatherapy session data that the personal computing device received from the set of aroma vapor dispensers. The user history data can also include or be linked with the particular aroma vapor(s) dispensed during each aroma therapy session, and the relative dispensing or blend percentages of such vapors, by way of the set of dispensing unit ID's and the aroma vapor dispensing control signals corresponding to the aromatherapy session. The aroma vapor dispensing management app can visually present user history data, e.g., in graphical form, to the system user as part of one or more aroma vapor dispensing user interfaces.

Finally, in some embodiments the aroma vapor dispensing management app can control the set of aroma vapor dispensing user interfaces to provide or present an aroma therapy session scheduling interface, which is configured for receiving user input directed to defining timing or schedule data corresponding to times or dates/times at which the set of aroma vapor dispensers is to automatically initiate one or more aromatherapy sessions in accordance with a set of aroma vapor dispensing control signals. The personal computing device can communicate such schedule data to the set of aroma vapor dispensing devices in association with the communication of aroma vapor dispensing control signals thereto.

Further aspects of aroma vapor dispensing systems in accordance with particular non-limiting representative embodiments of the present disclosure are described in detail hereafter, with reference to specific FIGS. included herewith.

Aspects of Representative Aroma Vapor Dispensing System Configurations

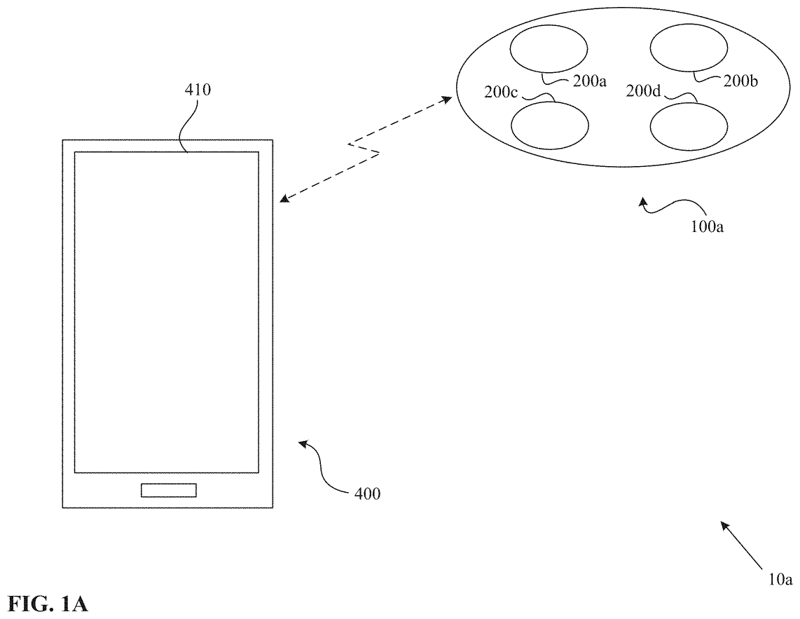

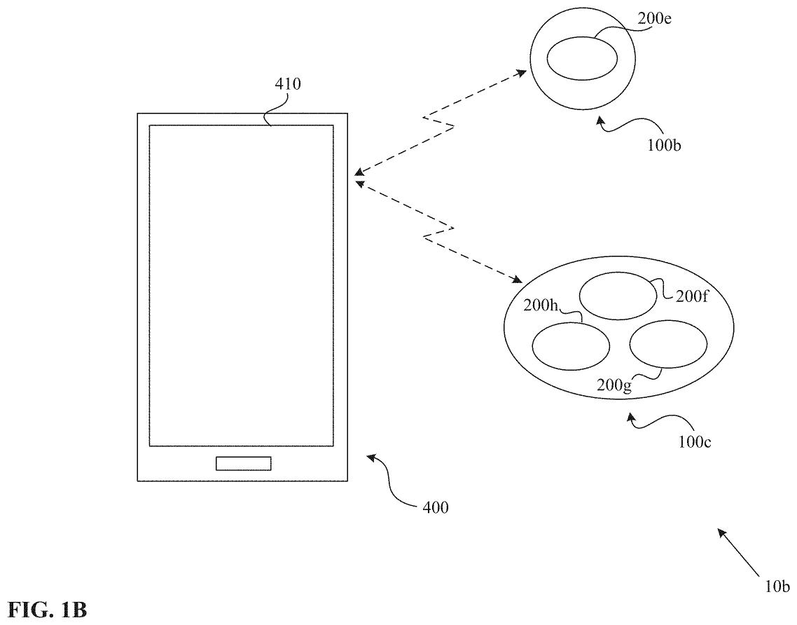

FIGS. 1A and 1B are schematic illustrations of particular non-limiting representative aroma vapor dispensing systems 10a,b in accordance with embodiments of the present disclosure. Each aroma vapor dispensing system 10a,b includes a set of aroma vapor dispensers 100 providing multiple concurrently-activatable aroma fluid reservoir units 200; and a personal computing device 400 having a touch screen display 410. The personal computing device 400 is configured for signal communication with the set of aroma vapor dispensers 100. In various embodiments, such signal communication is wireless, e.g., such as by way of signal exchange using communication units that support a Bluetooth™ wireless communication standard. In other embodiments, wireless or wire-based signal communication can occur in accordance with another wireless or wire-based communication standard, respectively. For instance, some embodiments are configured for wire-based signal communication, such as by way of Universal Serial Bus (USB) interfaces and a USB cable.

In a configuration such as that shown in FIG. 1A, the aroma vapor dispenser 100a is a single apparatus configured for carrying a predetermined maximum number of aroma fluid reservoir units 200, e.g., four aroma fluid reservoir units 200a-d. The aroma vapor dispenser 100a is configured for communicating a current set of reservoir unit IDs corresponding to these four aroma fluid reservoir units 200a-d to the personal communication device 400, for processing or analysis by an aroma vapor dispensing management app executing thereon. The aroma vapor dispensing management app can analyze the current set of reservoir unit IDs to determine which specific aroma fluid reservoir units 200a-d are presently engaged with the aroma fluid dispenser 100a, and are concurrently activatable thereby. The aroma vapor dispensing management app can further direct the generation of an aroma vapor dispensing user interface based on the current set of reservoir unit IDs, e.g., such that one or more of the color, geometry, and organization of the graphical elements presented thereby, and/or which graphical elements are selectable by the user, depends upon the current set of reservoir unit IDs. In response to user input received by way of the aroma vapor dispensing user interface, the aroma vapor dispensing management app can further determine a current set of aroma fluid vaporizing element control signals corresponding to the current set of reservoir unit IDs. After the personal computing device 400 communicates the current set of aroma vapor dispensing control signals to the aroma vapor dispenser 100a, the aroma vapor dispenser 100a can concurrently or simultaneously activate particular aroma fluid vaporizing elements in accordance with the aroma vapor dispensing control signals received from the personal computing device 400.

In other configurations, multiple distinct aroma vapor dispensers 100b,c can be present, each of which is configured for carrying at least one aroma fluid reservoir unit 200. For instance, in the configuration such as that shown in FIG. 1B, one aroma vapor dispenser 100b can be configured for carrying a single aroma fluid reservoir unit 200e; and another aroma vapor dispenser 100c can be configured for carrying multiple aroma fluid reservoir units 200f-h (e.g., in a manner analogous to that shown in FIG. 1A). Each such aroma vapor dispenser 100b,c is configured for communicating its current set of reservoir unit IDs to the personal computing device. The aroma vapor dispensing management app can analyze the sets of reservoir unit IDs from the distinct aroma vapor dispensers 100b,c as a current combined or composite set of reservoir unit IDs to determine which specific aroma fluid reservoir units 200e-h are presently engaged with these aroma fluid dispensers 100b,c, and are concurrently activatable/controllable under the direction of such aroma fluid dispensers 100b,c. The aroma vapor dispensing management app can further direct the generation of an aroma vapor dispensing user interface based on the current composite set of reservoir unit IDs., e.g., such that the color, geometry, and/or organization of the graphical elements presented thereby, and/or which graphical elements are selectable by the user, depends upon the current composite set of reservoir unit IDs. In response to user input received by way of the aroma vapor dispensing user interface, the aroma vapor dispensing management app can further determine a current composite set of aroma fluid vaporizing element control signals corresponding to the current composite set of reservoir unit IDs. After the personal computing device 400 communicates appropriate portions of the current composite set of aroma vapor dispensing control signals to each aroma vapor dispenser 100b,c, the aroma vapor dispensers 100b,c can concurrently activate particular aroma fluid vaporizing elements in accordance with the aroma vapor dispensing control signals received from the personal computing device 400.

Aspects of Representative Aroma Vapor Dispensers

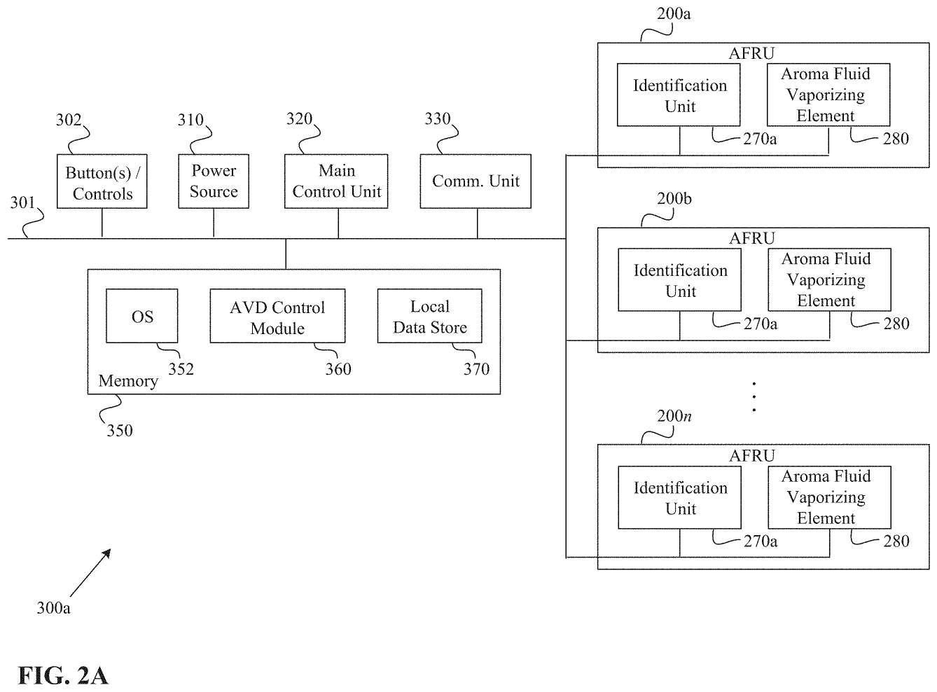

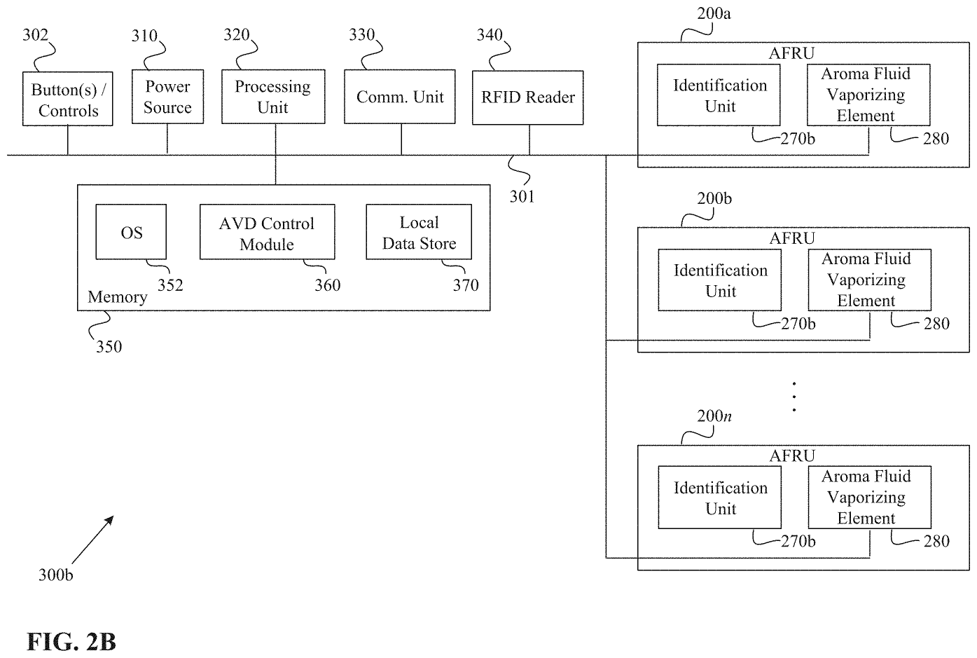

FIGS. 2A-2B are block diagram showing portions of aroma vapor dispenser electronic element configurations or architectures 300 in accordance with particular non-limiting representative embodiments of the present disclosure. As indicated in FIG. 2A, in an embodiment, an aroma vapor dispenser 100 has an electronic element configuration 300a that includes a set of buttons/controls 302, for instance, a set of multi-function controls including on/off button or switch; a power source 310 (e.g., a rechargeable battery); a main control unit 320 (e.g., a main microcontroller); a communication unit 330; and a memory 350. In some embodiments, an operating system (OS) resides in the memory 350, but other embodiments an OS is not present, e.g., depending upon the type of main control unit 320 present. The memory 350 also stores an aroma vapor dispenser (AVD) control module 360, which includes one or more program instruction sets executable by the main control unit 320. The memory 350 further includes a local data store and/or database 370, e.g., which stores (a) one or more sets of aroma fluid vaporizing apparatus, device, or element control signals, including a current set of aroma fluid vaporizing apparatus, device, or element control signals; and in some embodiments, (b) current/recent aromatherapy session data.

The aroma vapor dispenser's main control unit 320 is configured for selectively activating a set of aroma fluid reservoir units (AFRUs) 200a-n, such as different combinations among multiple aroma fluid reservoir units 200a-n, in accordance with the current set of aroma fluid vaporizing element control signals. Each AFRU 200a-n carries its own identification unit 270a, which stores a reservoir unit ID corresponding to the aroma fluid reservoir unit 200. Each AFRU 200a-n also carries an aroma fluid vaporizing element 280. In an embodiment such as that shown in FIG. 2A, each identification unit 270a includes or is a microcontroller, e.g., which is electrically couplable to the aroma vapor dispenser's power source 310 and main control unit 320 in association with engagement (e.g., mating engagement) of the aroma fluid reservoir unit 250 with the aroma vapor dispenser 100. Each AFRU's aroma fluid vaporizing element 280 is also couplable to the aroma vapor dispenser's power source 310 and main control unit 320 by way of such engagement.

Each element within the aroma vapor dispenser's electronic architecture 300 can be coupled to a set of signal lines or buses 301, in a manner readily understood by individuals having ordinary skill in the art.

FIG. 2B shows portions of another aroma vapor dispensing electronic configuration 300b, in which the identification unit 270a of each ARFU 200a-n includes or is an RFID tag, in which case the aroma vapor dispenser 100 includes an RFID reader 340 configured for communicating with each such identification unit 270a.

In a representative implementation, the main control unit 320 can be a TI BQ2022 (Texas Instruments Incorporated, Dallas, Tex. USA), which is a surface mount integrated circuit package having gull wing leads extending from each of four package sides, e.g., providing 48 pins in total, with 12 pins per side. The communication unit 330 can include a TI CC22540. The communication unit 330 can also include standard wire-based communication circuitry, such as micro-USB and/or other type of standard interface circuitry, e.g., by way of which the power source 310 can be recharged. Additionally or alternatively, the aroma vapor dispenser 100 can include a standard direct current (DC) charging interface, e.g., couplable or coupled to an alternating current (AC) adapter for recharging the battery 310 or powering the aroma vapor dispenser 100. In an implementation in which the identification unit 270 carried by each AFRU 200 includes or is a microcontroller, the microcontroller can be an STM 32F0300 (STMicroelectrronics, Geneva, Switzerland). In an implementation in which each AFRU 200 carries an RFID tag as its identification unit, the aroma vapor dispenser 100 can include an RFID reader/writer such as a SkyeRead™ M1 Mini (JADAK Technologies, Inc., North Syracuse, N.Y., USA), and the RFID tag can be a commercially-available RFID tag suitable for small or relatively small objects, with which the RFID reader/writer can communicate.

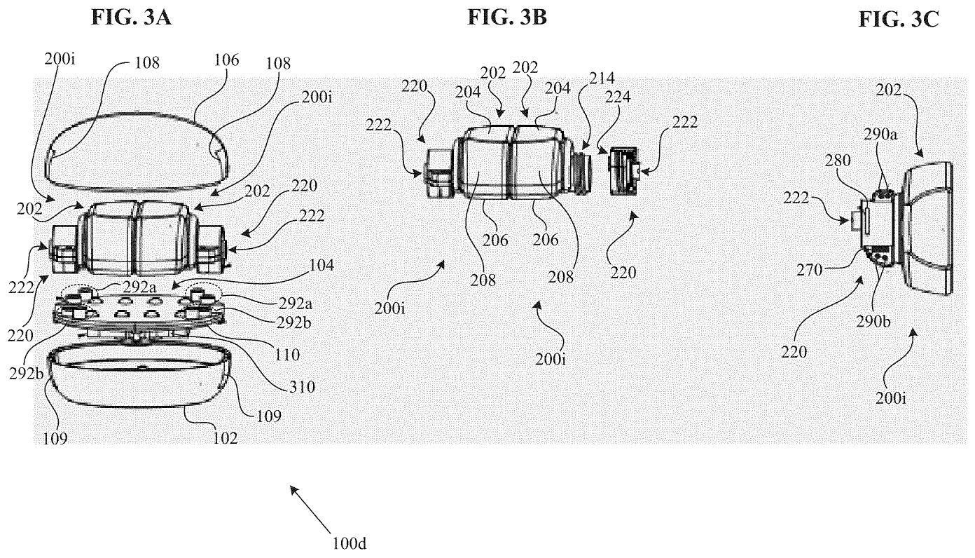

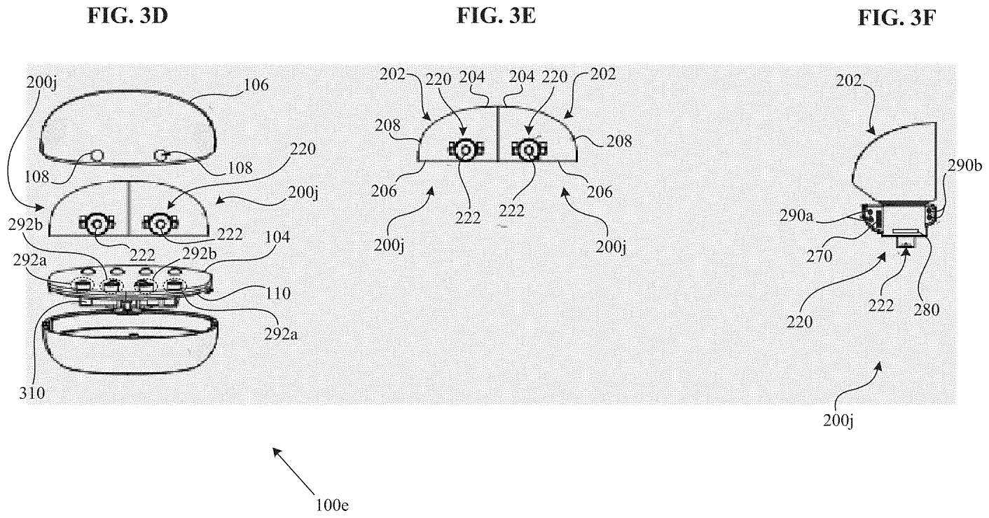

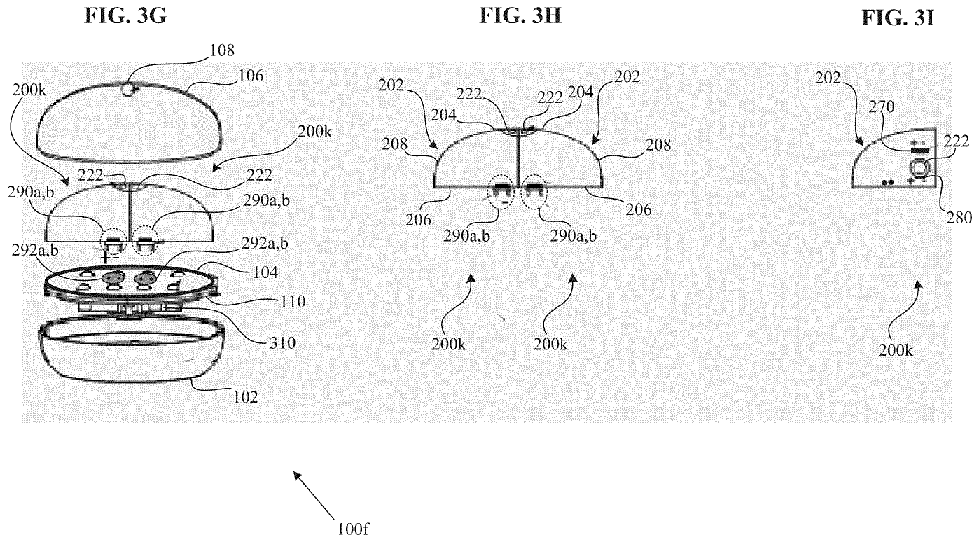

FIGS. 3A-3I are schematic illustrations of aroma vapor dispensers 100 in accordance with particular non-limiting representative embodiments of the present disclosure, e.g., which can dispense aroma vapors by way of aroma fluid reservoir units 200 that can contain aroma substances such as essential oils that are present, diluted, or distributed in a carrier fluid (e.g., water). For purpose of illustration and simplicity, the aroma vapor dispenses 100 shown in FIGS. 3A-3I are configured for carrying two aroma fluid reservoir units 200. Notwithstanding, individuals having ordinary skill in the art will understand that structural aspects of aroma vapor dispensers 100 configured for carrying a single or more than two aroma fluid reservoir units 200 are analogous to those described hereafter for the representative embodiments shown in FIGS. 3A-3I.

FIGS. 3A-3C are schematic illustrations of an aroma vapor dispenser 100d in accordance with a first non-limiting representative embodiment of the present disclosure. With respect to FIG. 3A, in an embodiment the aroma vapor dispenser 100d includes a housing providing a bottom or base portion 102; a top or cover portion 106; and an intermediate portion or support member 104 disposable or disposed therebetween. The aroma vapor dispenser's battery 310 can be disposed below a first or underside of the intermediate support member 104, e.g., by way of structural coupling to the intermediate support member 104 or the base portion 102. A circuit board 110 can be carried by the underside of the intermediate support member 104 above the battery 310, such as by mounting of the circuit board 110 to the underside of the intermediate support member 104. The circuit board 110 can carry the aroma vapor dispenser's main control unit 320, communication unit 330, and memory 350. Additionally, the set of buttons/controls 302 can be carried by the base portion 102, e.g., beneath a bottom surface thereof that is intended to rest against a support surface such as a table or desk while the aroma vapor dispenser 100d is in use.

An upper side of the intermediate support member 104 can be configured for carrying the aroma fluid reservoir units 200i, such that each aroma fluid reservoir unit 200i is removably engageable with the intermediate support member's upper side.

FIG. 3B illustrates particular structural aspects of two aroma fluid reservoir units 200i in accordance with an embodiment of the present disclosure, each of which has an identical structure. In an embodiment, each aroma fluid reservoir unit 200i includes a receptacle body 202 providing an internal cavity or chamber therein configured for holding up to a predetermined maximum volume of an aroma fluid. In general, the receptacle body 202 includes an upper or top surface or side 204; a lower or bottom surface or side 206; and a set of peripheral surfaces 208 extending between the top surface 204 and the bottom surface 206. The receptacle body 202 can include or be formed of one or more types of materials, e.g., one or more polymer materials that are inert to aroma fluids/essential oils, a metal such as stainless steel, glass, or another material. In various embodiments, the lower surface 206 of the receptacle body 202 is supported over or by the upper surface of the intermediate support member 104 when the aroma fluid reservoir unit 200 is engaged therewith.

The aroma fluid reservoir unit 200i also includes a removable cap structure 220, e.g., a screw-on cap structure 220, which in various embodiments is configured for engaging (e.g. mating screw-on engagement) with a portion of the receptacle body 202 in a manner that establishes a water-tight or hermetic seal, such that the aroma fluid reservoir unit 200i can be disposed in essentially any orientation without aroma fluid leakage from the receptacle body 202 while the cap structure 220 is properly matingly engaged with the receptacle body 202. For instance, the receptacle body can carry a set of male screw threads 214, and the cap structure 220 can carry a set of counterpart female screw threads 224 configured for screw-type engagement therewith; and the cap structure 220 can include a washer therein that is circumferentially disposed about the female screw threads 224 to enable the formation of a hermetic or water-tight seal with the receptacle body 202. The cap structure 220 also includes an exit aperture 222 by which aroma vapor can exit the cap structure 210 and is dispensed from the aroma fluid reservoir unit 200i upon vaporization of a portion of the aroma fluid carried within the receptacle body's cavity.

FIG. 3C is a plan view showing portions of a cap structure 220 as viewed from an underside thereof in accordance with an embodiment of the present disclosure. In an embodiment, the cap structure 220 carries the aroma fluid reservoir unit's aroma fluid vaporizing element 280, such that the aroma fluid vaporizing element 280 is exposed to aroma fluid within the receptacle body's cavity when the cap structure 220 is matingly engaged therewith. In various embodiments, the aroma fluid vaporizing element 280 includes or is a piezoelectric transducer. The cap structure 220 includes a first pair of electrical pins 290a by which the aroma fluid vaporizing element 280 can be electrically coupled to and activatable by the main control unit 320.

In various embodiments, the cap structure 220 is engageable with the receptacle body 202 such that the aroma fluid vaporizing element 280 is positionable or positioned closer to the lower surface 206 of the receptacle body 202 than the top surface 204 of the receptacle body 202, e.g., generally near or proximate to the receptacle body's lower surface 206. Such a positioning of the aroma fluid vaporizing element 280 can ensure that when the aroma vapor dispenser 200i is in an upright position and the aroma fluid within the receptacle body 202 is being vaporized, the aroma fluid level (or height) within the receptacle body 202 progressively decreases over time toward the receptacle body's lower surface 206, and the aroma fluid vaporizing element 280 remains exposed to the aroma fluid for as long as possible throughout the progressive decrease(s) in aroma fluid level until the receptacle body 202 is very nearly or essentially empty.

Depending upon embodiment details, a predetermined portion of the aroma fluid reservoir unit 200i, such as a predetermined portion of the cap structure 220 or a predetermined portion of the receptacle body 202 can carry the aroma fluid reservoir unit's identification unit 270. In the embodiment shown in FIGS. 3A-3C, the identification unit 270 is disposed at a predetermined location within the cap structure 220. The cap structure 220 includes a second pair of electrical pins 290b by which the aroma fluid vaporizing element 280 can be electrically coupled to and activatable by the main control unit 320.

With reference again to FIG. 3A, the intermediate support member 104 includes a counterpart first pair of electrical pin receivers 292a configured for mating engagement with cap structure's first pair of electrical pins 290a corresponding to the aroma fluid vaporizing element; and a second pair of electrical pin receivers 292b configured for mating engagement with the second pair of electrical pins 290b corresponding to the cap structure's identification unit 270, thereby enabling electrical coupling of the identification unit 270 to the aroma vapor dispenser's main control unit 320.

As indicated in FIG. 3A, the aroma vapor dispenser's first and second pairs of electrical pin receivers 292a,b are located at predetermined positions or sites relative to the intermediate support member 104. These predetermined positions or sites are configured for mating engagement with counterpart electrical pins 290a,b carried by the aroma fluid reservoir units 200.

These predetermined positions or sites therefore correspond to the physical sites or positions at or in which the user can load and unload aroma vapor reservoir units 200 from the aroma vapor dispenser's housing. Consequently, when a particular aroma fluid reservoir unit 200 that has been loaded on or in the aroma dispenser's housing communicates its reservoir unit ID to the main control unit 320, the main control unit 320 or the aroma vapor dispenser control module 360 executing thereon can recognize the specific physical site or position on or in the aroma vapor dispenser's housing at which this aroma fluid reservoir unit 200 resides. Furthermore, after the main control unit 320 receives a current set of aroma vaporizing element control signals linked with the current set of reservoir IDs from the personal computing device 400, the main control unit 400 can activate the aroma vaporizing elements 270 within the appropriate or correct aroma vapor reservoir units 200 in accordance with the current set of aroma vaporizing element control signals regardless of the physical housing positions or sites they occupy.

The housing's cover portion 106 is engageable with the intermediate support member 104 and/or the base portion 102, and surrounds or overlays at least portions of the aroma fluid reservoir units 200i that are engaged with the aroma vapor dispenser 100d. In various embodiments, the cover portion 106 includes a plurality of cutouts, recesses, or openings 108 therein corresponding to the positions and/or orientations of the exit apertures 222 on the aroma fluid reservoir units 200i that the aroma vapor dispenser 100d carries. Each such cutout 108 corresponds to a spatial region at which an aroma vapor dispensed by a given aroma fluid reservoir unit 200i can travel away from the aroma vapor dispenser 100d into the aroma vapor dispenser's external environment. Multiple aroma vapors that travel away from such cutouts 108 and can blend each other in the vicinity of the aroma vapor dispenser 100d. In several embodiments, the cover portion 106 includes upper cutouts 108, and the base portion 102 can includes counterpart lower cutouts 109, which align with the upper cutouts 108, such that the aroma fluid reservoir unit exit apertures 222 are disposed between the upper cutouts 108 and the lower cutouts 109.

With respect to overall aroma vapor dispenser dimensions, in embodiments such as shown in FIGS. 3A-3I, the aroma vapor dispenser 100 can be defined to have a maximum length along a lengthwise centerline thereof, which is greater than a maximum width along a widthwise centerline thereof, where the lengthwise and widthwise centerlines are perpendicular to each other. In the embodiment of FIGS. 3A-3C, the aroma fluid reservoir units' exit apertures 222 and the cutouts 108 are aligned at least approximately coincident with or are disposed at least approximately parallel to the lengthwise centerline of the aroma vapor dispenser 100d, such that concurrently dispensed aroma vapors initially travel away from side surfaces of the aroma vapor dispenser 100d at a first separation distance from each other relative to the length of the aroma vapor dispenser's housing. However, other exit aperture-cutout alignments are possible, which may influence, affect, or enhance aroma vapor blending.

FIGS. 3D-3F are schematic illustrations of an aroma vapor dispenser 100e in accordance with a second non-limiting representative embodiment of the present disclosure, in which the exit apertures 222 provided by a pair of aroma fluid reservoir units 200g and the cutouts 108 provided by the housing's cover portion 106 are aligned at least approximately coincident with or are disposed at least approximately parallel to the widthwise centerline of the aroma vapor dispenser 100e, such that concurrently dispensed aroma vapors initially travel away from side surfaces of the aroma vapor dispenser 100d at a second separation distance from each other relative to the width of the aroma vapor dispenser's housing, where the second separation distance is less different than, e.g., less than, the first separation distance.

FIGS. 3G-3I are schematic illustrations of an aroma vapor dispenser 100f in accordance with a third non-limiting representative embodiment of the present disclosure, in which the exit apertures 222 provided by a pair of aroma fluid reservoir units 200g and the cutouts 108 provided by the housing's cover portion 106 face in an upward or generally upward direction and are disposed proximate or adjacent to each other in a manner that is aligned at least generally perpendicular to each of the lengthwise and widthwise axes of the aroma vapor dispenser 100f, such that concurrently dispensed aroma vapors initially travel upward away from the aroma vapor dispenser's cover portion 106.

Aspects of Representative Personal Computing Devices

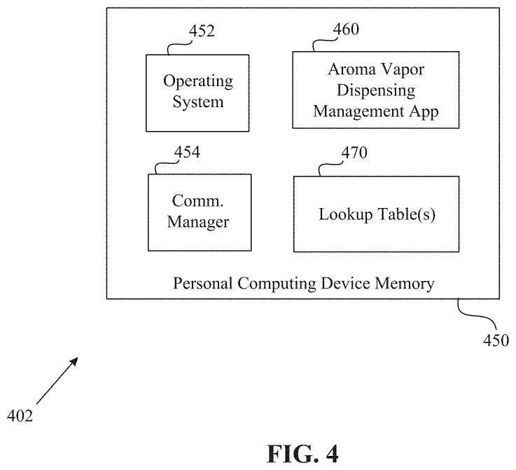

FIG. 4 is a block diagram showing portions of a personal computing device memory contents configuration or architecture 402 in accordance with a particular non-limiting representative embodiment of the present disclosure. In an embodiment, a personal computing device 400 includes a memory 450 in which program instruction sets executable by the personal computing device's processing unit(s) reside. Such program instructions sets include an OS 452; a communication manager 454 configured for managing wireless and/or wire-based signal communication processes or operations; and an aroma vapor dispensing management app 460. The memory 450 further can further store one or more lookup tables 470, which contain mappings of different combinations of reservoir unit IDs to particular relative combinations of aroma fluid vaporizing element control signals. The aroma vapor dispensing management app 460 can access such lookup tables 470 in association with mapping or converting user input directed to particular user selectable graphical elements to relative or interdependent aroma fluid vaporizing element control signals, which correspond to particular relative or interdependent aroma vapor blend ratios.

Aspects of Representative Aroma Vapor Dispensing User Interfaces

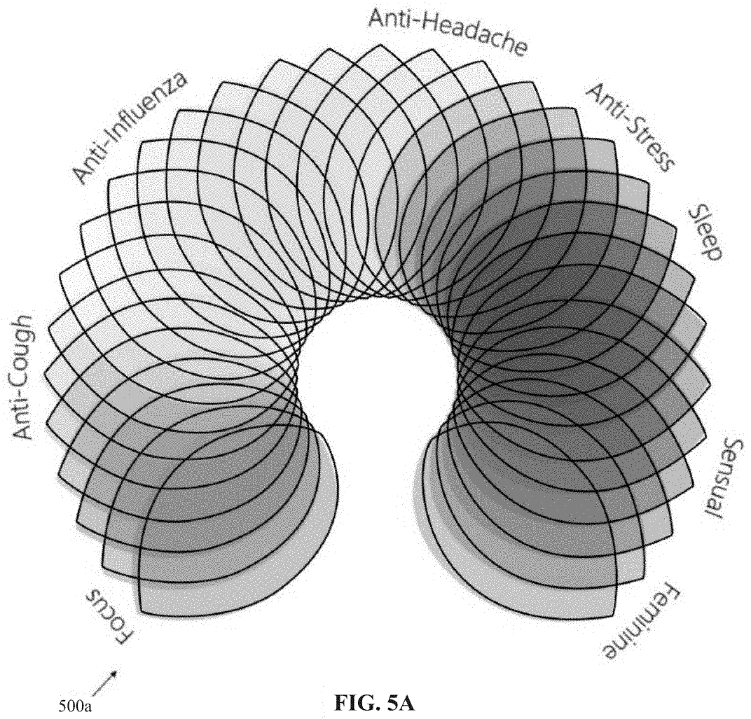

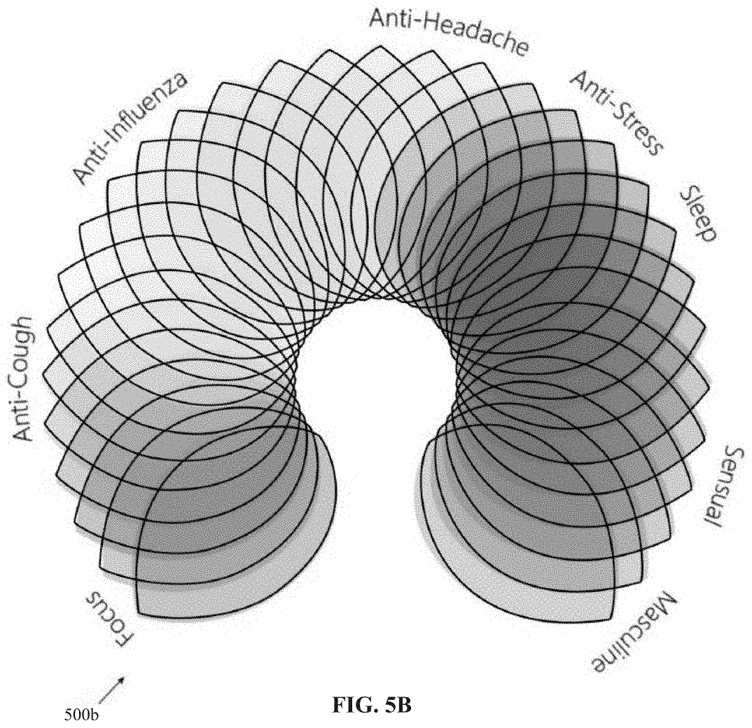

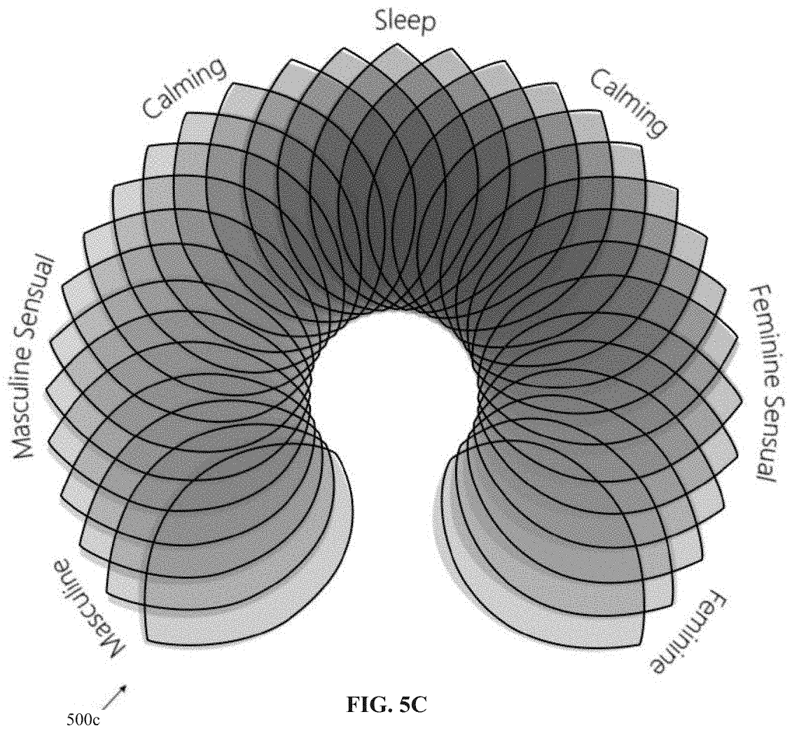

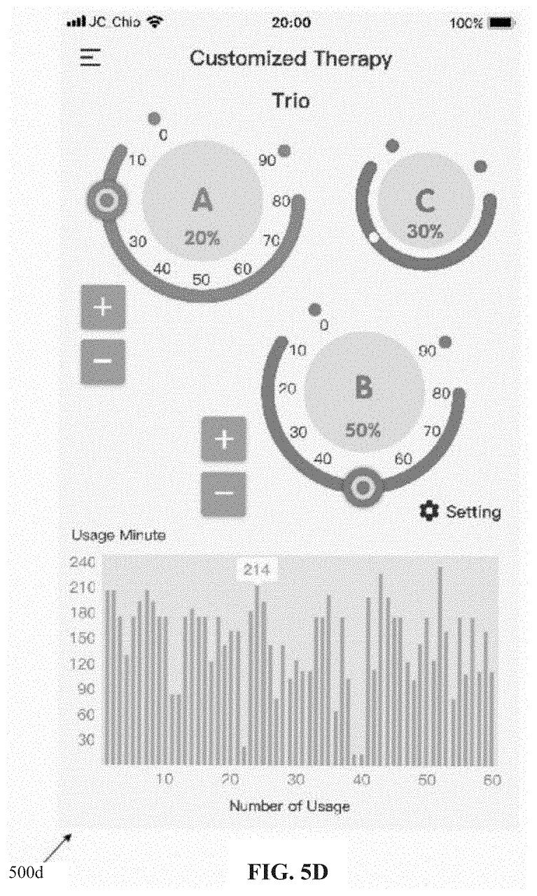

FIG. 5A is an image of a first color wheel 500a provided by a set of aroma vapor dispensing user interfaces in accordance with a non-limiting representative embodiment of the present disclosure. In an embodiment, the first color wheel 500a has a shape that at least generally corresponds or corresponds to a circle or partial circle. A first or left extremity of the first color wheel 500a includes graphical elements displayed in accordance with a first color corresponding to a first color, e.g., red, of a visual spectrum, such as one or more portions of the visible optical spectrum; and a second or right extremity of the first color wheel 500a includes graphical elements displayed in accordance with a second color, e.g., violet, of the visual spectrum. Along a display device path from the left extremity toward and to the right extremity, the first color wheel 500a includes graphical elements that are displayed with colors that progressively change in accordance with the visual spectrum under consideration.

Visual identifiers in the form of textual labels are presented at particular locations relative to the periphery of the first color wheel 500a. Such labels can include “Focus,” “Anti-Cough,” “Anti-Influenza,” “Anti-Headache,” “Anti-Stress,” “Sleep,” “Sensual,” and “Feminine,” which indicate biological or physiological effects typically associated with different individual aroma vapors and different aroma vapor combinations that can be generated by way of vaporization of the aroma fluids contained within the aroma fluid reservoir units 200 corresponding to a current set of reservoir unit IDs. Successive labels from “Focus” to “Sensual” can be associated with colors and color blends from red to blue in the first color wheel 500a; and the label “Feminine” can be associated with shades of pink colors in the first color wheel 500a. Certain of such labels can be presented adjacent to portions of the first color wheel 500a that correspond to primary colors (e.g., red, green, and blue), which can indicate that in response to user selection of graphical elements corresponding to such primary colors, the aroma vapor dispenser(s) 100 will output a specific individual aroma vapor from one of its aroma fluid reservoir units 200 rather than a blend of aroma vapors from multiple aroma fluid reservoir units 200. User selection of graphical elements between primary colors (e.g., orange colored graphical elements, which corresponds to a visual color combination of red and yellow) can indicate that in response to user selection of graphical elements corresponding to blends of primary colors, the aroma vapor dispenser(s) 100 will output a specific blend of aroma vapors from two or more of its aroma fluid reservoir units 200 rather than a single aroma vapor from a single aroma fluid reservoir unit 200.

As indicated above, the particular graphical elements available for user selection, e.g., a current plurality of user selectable graphical elements in the first color wheel 500a, can depend or vary based upon the specific individual reservoir unit IDs within the current set of reservoir unit IDs. For instance, if a first current set of reservoir unit IDs indicates that only a single aroma fluid reservoir unit 200 associated with a particular type of aroma fluid is present within the aroma vapor dispenser 100, then only specific user selectable graphical elements, e.g., a first (sub)set of graphical elements, within the first color wheel 500a will be associatable with user input, e.g., where such user selectable graphical elements are displayed or visually perceivable as a set of colors within a predetermined color shade range (e.g., light blue to dark blue) that is much narrower than the complete distribution of colors within the first color wheel 500a. Alternatively, if a second current set of reservoir unit IDs indicates that two aroma fluid reservoir units 200 are present within the aroma vapor dispenser 100, each of which contains a different particular aroma fluid, then additional or other graphical elements within the first color wheel 500a will be associatable with user input.

Further to the foregoing, color wheels containing different colors, and/or different labels associated with such color wheels, can be displayed based on the specific individual reservoir unit IDs within the current set of reservoir unit IDs. For instance, FIG. 5B is an image of a second color wheel 500b provided by a set of aroma vapor dispensing user interfaces in accordance with a non-limiting representative embodiment of the present disclosure, which could be displayed instead of the first color wheel 500a depending upon the reservoir unit IDs present in the current set of reservoir unit IDs. The second color wheel 500b includes the label “Masculine” instead of “Feminine,” where the label “Masculine” is associated with shades of violet instead of shades of pink.

FIG. 5C is an image of a third color wheel 500c provided by a set of aroma vapor dispensing user interfaces in accordance with a non-limiting representative embodiment of the present disclosure. The third color wheel 500c has a leftmost label of “Masculine,” and successive labels around the third color wheel 500c in a clockwise direction including “Masculine Sensual,” “Calming,” “Sleep,” “Calming,” “Feminine Sensual,” and “Feminine.” The label “Masculine” can be displayed proximate or adjacent to graphical elements within the third color wheel 500c having shades of violet, and the label “Feminine” can be displayed proximate or adjacent to graphical elements within the third color wheel 500c having shades of pink. Graphical elements along the third color wheel 500c between the label “Masculine” and the label “Feminine” can progressively vary in color. The left half of the third color wheel 500c, the graphical elements therein, and the labels thereof can correspond to biological or physiological effects relevant to males for particular individual aroma vapors and/or aroma vapor blends; and the right half of the third color wheel 500c, the graphical elements therein, and the labels thereof can correspond to biological or physiological effects relevant to females for certain individual aroma vapors and/or aroma vapor blends.

FIG. 5D is an image of an aroma vapor dispensing interface 600 provided by a set of aroma vapor dispensing user interfaces in accordance with a non-limiting representative embodiment of the present disclosure, which corresponds to aroma fluid reservoir units containing user-customized aroma fluid contents, and which provides user aroma therapy history information. In an embodiment such as shown in FIG. 6D, the current set of reservoir unit IDs includes three distinct or different reservoir unit IDs (e.g., three differently valued reservoir unit IDs), each of which corresponds to a distinct or different aroma vapor. Three geometric shapes labeled as “A”, “B”, and “C” can be displayed corresponding to each of these three different reservoir unit IDs. In response to (a) user input directed to user selectable graphical elements of geometric shapes “A” and “B”, thereby indicating relative aroma vapor blend percentages of an aroma vapor corresponding to geometric shape “A” as established by one of the current reservoir unit IDs with a different aroma vapor corresponding to geometric shape “B” as established by another one of the current reservoir unit IDs, (b) the aroma vapor dispensing management app can automatically determine a relative aroma vapor blend percentage for an aroma vapor corresponding to geometric shape “C” as established by yet another different current reservoir unit ID, where the individual aroma vapor blend percentages corresponding to geometric shapes “A,” “B,” and “C” total to 100%. Among aroma vapor blend percentages corresponding to combinations or blends of the aroma vapor corresponding to geometric shape “A” and the aroma vapor corresponding to geometric shape “B,” an overall or total blend percentage cannot exceed 100% (and when it equals 100%, no graphical objects within the geometric shape corresponding to aroma vapor “C” are associatable with user input, and hence the blend percentage corresponding to this aroma vapor equals 0%). Similarly, for aroma vapor blend percentages corresponding to combinations or blends of the aroma vapor corresponding to geometric shape “A,” the aroma vapor corresponding to geometric shape “B,” and the aroma vapor corresponding to geometric shape “C,” an overall or total blend percentage cannot exceed 100%.

The aroma vapor dispensing management app can automatically determine a user selectable blend percentage range for the aroma vapor corresponding to geometric shape “A” based on its corresponding reservoir unit ID, and can auto-adjust the user selectable blend percentage range for the aroma vapor corresponding to geometric shape “B” based on its corresponding reservoir unit ID. The aroma vapor dispensing management app can display most-recently determined aroma vapor blend percentages corresponding to geometric shapes “A,” “B,” and “C” on a dynamic basis, e.g., on the fly in response to user input.

Table 1 below provides a representative set of aroma fluid or aroma vapor blend percentages that can be provided in response to user selection of graphical elements corresponding to 10%, 20%, 30%, 40%, 50%, 60%, 70%, 80%, and 90% visual labels presented at different “clock face” positions or “clock positions” relative to or around portions of geometric shapes “A” and “B.”

For geometric shapes “A” and “B,” the aroma vapor dispensing user interface can provide a standard type of graphical control, e.g., a slider control, that is graphically displaceable about or along portions of the geometric shape in response to user input, thereby enabling aroma vapor blend ratio selections in response to user input, including blend ratio selections/adjustments in response to user input directed to user-selectable graphical elements located between such “clock positions.”

TABLE 1
Aroma Vapor Blend Percentages Associated
with Visual Labels Displayed at “Clock Positions”
on the Aroma Vapor Dispensing User Interface

| “Clock Position” | | | |
|---|---|---|---|
| on User Interface | A % | B % | C % |
| 10 am | 10 | 10-80 | 80-10 |
| 9 am | 20 | 10-70 | 70-10 |
| 8 am | 30 | 10-60 | 60-10 |
| 7 am | 40 | 10-50 | 50-10 |
| 6 am | 50 | 10-40 | 40-10 |
| 5 am | 60 | 10-30 | 30-10 |
| 4 am | 70 | 10-20 | 20-10 |
| 3 am | 80 | 10 | 10 |

Particular embodiments in accordance with the present disclosure have been described in detail herein for purpose of illustration to aid understanding. While particular aspects of such embodiments are detailed herein, individuals having ordinary skill in the relevant art can make one or more modifications to some or each of such embodiments. The embodiments described herein and such modifications thereto fall within the scope of the present disclosure, which is limited only by the following claims.

1. An aroma fluid vapor dispensing system, comprising:
a set of aroma vapor dispensers providing a plurality of aroma fluid reservoir units, and configured for controlling aroma vapors output from the plurality of aroma fluid reservoir units in accordance with a plurality of aroma vapor dispensing control signals, each aroma fluid reservoir unit configured for carrying an aroma fluid and transforming the aroma fluid into an aroma vapor in accordance with an aroma vapor dispensing control signal, wherein each aroma vapor dispenser comprises:
a power source;
a main control unit electrically coupled to the power source;
a communication unit electrically coupled to the main control unit, and configured for signal communication with an electronic device external to the aroma vapor dispenser;
a set of aroma fluid reservoir units, each aroma fluid reservoir unit comprising:
a receptacle having a cavity therein for carrying an aroma fluid volume; and
an aroma fluid vaporizing element fluidically coupled to the cavity, the aroma fluid vaporizing element electrically couplable to and activatable by way of the main control unit;
a set of identification units storing a set of electronically-readable reservoir unit identifiers (IDs) corresponding to the set of aroma fluid reservoir units; and
a memory electrically coupled to the main control unit, and configured for storing an aroma vapor dispensing control signal corresponding to each aroma fluid vaporizing element within the set of aroma fluid reservoir units, and which corresponds to an electrical signal level at which the aroma fluid vaporizing element is driven during aroma vapor dispenser operation; and
a personal computing device external to the set of aroma vapor dispensers, comprising:
a power supply;
a processing unit coupled to the power supply;
a set of user interface devices including a display device and a user input device coupled to the processing unit;
a communication unit coupled to the processing unit and configured for signal communication with each aroma vapor dispenser within the set of aroma vapor dispensers;
a memory coupled to the processing unit, wherein the memory includes an operating system and an aroma vapor dispensing management app, wherein the aroma vapor dispensing management app comprises program instructions which when executed by the processing unit perform aroma vapor dispensing management operations comprising:
establishing signal communication between the personal computing device and the set of aroma vapor dispensers;
receiving a plurality of current reservoir unit IDs from the set of aroma vapor dispensers, each reservoir unit ID within the plurality of current reservoir unit IDs associated with a particular aroma fluid reservoir unit within the plurality of aroma fluid reservoir units;
automatically generating and presenting on the display device an aroma vapor dispensing user interface that provides visual information content including a plurality of graphical elements, at least some of which are user-selectable, wherein at least one of visual information content colors and graphical element user selectablity depends upon the plurality of current reservoir unit IDs, wherein each user-selectable graphical element interface is correlatable with an electronic signal level at which an aroma fluid vaporizing element of a particular aroma fluid receptacle unit within the plurality of aroma fluid reservoir units can be driven;
receiving a user selection corresponding to a first user-selectable graphical element within the aroma vapor dispensing user interface by way of the set of user interface devices;
dynamically determining a first plurality of aroma vapor dispensing control signals in response to user selection of the first user-selectable graphical element, each aroma vapor dispensing control signal within the first plurality of aroma vapor dispensing control signals corresponding to a particular aroma fluid receptacle unit within the plurality of aroma fluid receptacle units; and
communicating the first plurality of aroma vapor dispensing control signals to the set of aroma vapor dispensers.

2. The aroma fluid vapor dispensing system of claim 1, wherein the personal computing device comprises one of a smart wrist-worn device, a smart head-mounted apparatus, a smartphone, a tablet computing device, a phablet computing device, a laptop computer, and a desktop computer.

3. The aroma fluid vapor dispensing system of claim 1, wherein:
the receptacle of each aroma fluid reservoir unit is removable/replaceable and re-fillable; and
each aroma fluid reservoir unit further comprises a removable lid or cap configured for re-sealable water-tight engagement with the aroma fluid reservoir unit's receptacle.

4. The aroma fluid dispensing system of claim 1, wherein for each aroma vapor dispensing unit, each distinct aroma fluid reservoir unit thereof is configured for carrying its own identification unit.

5. The aroma fluid dispensing system of claim 4, wherein:
each aroma fluid dispenser further comprises a radio frequency identification (RFID) reader coupled to the aroma fluid dispenser's main control unit; and
the identification unit of each aroma fluid reservoir unit comprises an RFID tag storing the reservoir unit ID associated with the aroma fluid reservoir unit, which is communicable to the RFID reader.

6. The aroma fluid dispensing system of claim 4, wherein the identification unit of each aroma fluid reservoir unit comprises a microcontroller carried by the aroma fluid reservoir unit, which is configured for communicating the reservoir unit ID associated with the aroma fluid reservoir unit to the main control unit.

7. The aroma fluid vapor dispensing system of claim 3, wherein for each aroma fluid reservoir unit:
the receptacle thereof includes a bottom surface, a top surface, and at least one side interior surface extending therebetween;
the aroma fluid vaporizing element thereof is disposed closer to the bottom surface of the receptacle; and
the bottom surface of the receptacle is configured to reside closer to the surface of the earth than the top surface of the receptacle during activation of the aroma fluid vaporizing element, such that a level of an aroma fluid carried within the aroma fluid reservoir unit's receptacle progressively decreases over time toward the bottom surface of the receptacle.

8. The aroma fluid vapor dispensing system of claim 1, wherein:
each reservoir unit ID within the plurality of current reservoir unit IDs received by the personal computing device is associated with a distinct aroma fluid reservoir unit within the plurality of aroma fluid reservoir units and a particular aroma fluid contained therein that correspondingly produces a particular aroma vapor when vaporized;
different user-selectable graphical elements presented by the aroma vapor dispensing user interface correspond to different blend ratios among two or more aroma vapors that are simultaneously dispensable by the plurality of aroma fluid reservoir units; and
the first plurality of aroma vapor dispensing control signals corresponds to a specific blend ratio among a first plurality of aroma vapors dispensable by way of concurrent activation of a first plurality of aroma fluid vaporizing elements within the plurality of aroma fluid reservoir units in accordance with the first plurality of aroma vapor dispensing control signals.

9. The aroma fluid vapor dispensing system of claim 8, wherein:
each aroma fluid vaporizing element is drivable by way of an aroma vapor dispensing control signal corresponding to an electrical signal level between 0% and 100% of a maximum electrical signal level; and
dynamically determining the first plurality of aroma vapor dispensing control signals comprises establishing each aroma fluid vapor dispensing control signal within the first plurality of aroma vapor dispensing control signals on a relative basis with respect to each other aroma fluid vapor dispensing control signal within the first plurality of aroma vapor dispensing control signals, such that a mathematical sum corresponding to the individual fluid vapor dispensing control signal within the first plurality of aroma vapor dispensing control signals represents 100% of the maximum electrical signal level.

10. The aroma fluid vapor dispensing system of claim 1, wherein the plurality of user-selectable graphical elements corresponds to a plurality of different colors within portions of the visible optical spectrum.

11. The aroma fluid vapor dispensing system of claim 10, wherein the plurality of user-selectable graphical elements is presented as portions of one of (a) a color wheel, (b) a color palette, and (c) color coded geometric shapes.

12. The aroma fluid vapor dispensing system of claim 11, wherein:
the aroma vapor dispensing user interface further comprises a plurality of visual identifiers presented at predetermined display device coordinates or locations relative to the plurality of user-selectable graphical elements; and
the plurality of visual identifiers comprises at least one of textual, symbolic, and iconic visual content.

13. The aroma fluid vapor dispensing system of claim 12, wherein the plurality of visual identifiers indicates a plurality of intended or expected biological or physiological effects corresponding to exposure of a living mammal to different blend ratios among two or more aroma vapors providable by the plurality of aroma fluid reservoir units when the two or more aroma vapors are concurrently dispensed by the plurality of aroma fluid reservoir units.

14. The aroma fluid vapor dispensing system of claim 12, wherein the plurality of visual identifiers includes a first identifier associated with a first primary color corresponding to a first subset of graphical elements, a distinct second identifier associated with a second primary color corresponding to a distinct second subset of graphical elements, and a distinct third identifier associated with a third primary color corresponding to a distinct third subset of graphical elements.

15. The aroma fluid vapor dispensing system of claim 14, wherein the plurality of identifiers further includes a fourth identifier corresponding to a gender and associated with a distinct fourth subset of graphical elements.

16. The aroma fluid vapor dispensing system of claim 15, wherein the first, second, third, and fourth visual identifiers are positioned at different display device coordinates or locations relative to portions of portions of a color spectrum provided by the plurality of graphical elements.

17. The aroma fluid vapor dispensing system of claim 1, wherein the aroma vapor dispensing user interface further provides a graphical representation of user aromatherapy session history data over time.

18. The aroma fluid vapor dispensing system of claim 1, wherein:
each reservoir unit ID within the plurality of current reservoir unit IDs is associated with a particular type of aroma fluid carried by the aroma fluid reservoir unit corresponding to the reservoir unit ID; and
the aroma vapor dispensing management app includes program instructions which when executed by the processing unit perform further aroma vapor dispenser management operations comprising:
analyzing the plurality of current reservoir unit IDs;
determining whether the aroma fluid reservoir units carry identical or different aroma fluids based on the plurality of current reservoir unit IDs; and at least one of:
(a) adjusting the number of user-selectable graphical elements provided by the aroma vapor dispensing user interface if two or more aroma fluid reservoir units carry identical aroma fluids; and
(b) establishing a temporally alternating activation condition for the two or more aroma fluid reservoir units carrying identical aroma fluids, in accordance with equal valued aroma vapor dispensing control signals for the two or more aroma fluid reservoir units carrying identical aroma fluids.

19. The aroma fluid vapor dispensing system of claim 1, wherein the set of aroma vapor dispensers comprises a first aroma vapor dispenser that carries a first aroma fluid reservoir unit, and a physically separate second aroma vapor dispenser that carries a second aroma fluid reservoir unit.

20. The aroma fluid vapor dispensing system of claim 1, wherein the set of aroma vapor dispensers comprises a first aroma vapor dispenser configured for simultaneously carrying each of a first aroma fluid reservoir unit and a second aroma fluid reservoir unit.

21. The aroma fluid dispensing system of claim 20, wherein:
the first aroma vapor dispenser further comprises a holder or housing configured for carrying each of the power source, the main control unit, the communication unit, and the memory;
the first aroma fluid reservoir unit carries a first identification unit storing the first electronically-readable ID;
the second aroma fluid reservoir unit carries a second identification unit storing the second electronically-readable ID; and
the holder or housing is configured for removable mating engagement of the first and second aroma fluid reservoir units therewith.

22. An aroma vapor dispenser comprising:
a power source;
a main control unit electrically coupled to the power source;
a communication unit electrically coupled to the main control unit, and configured for signal communication with an electronic device external to the aroma vapor dispenser;
a set of aroma fluid reservoir units, each aroma fluid reservoir unit comprising:
a receptacle having a cavity therein for carrying an aroma fluid volume; and
an aroma fluid vaporizing element fluidically coupled to the cavity, the aroma fluid vaporizing element electrically couplable to and activatable by way of the main control unit;
a set of identification units storing a set of electronically-readable reservoir unit identifiers (IDs) associated with the set of aroma fluid reservoir units, wherein the set of reservoir unit IDs is electromagnetically or electronically communicable to the main control unit; and
a memory electrically coupled to the main control unit, and configured for storing an aroma vapor dispensing control signal corresponding to each aroma fluid vaporizing element within the set of aroma fluid reservoir units, and which corresponds to an electrical signal level at which the aroma fluid vaporizing element is driven during aroma vapor dispenser operation.

23. The aroma vapor dispenser of claim 22, wherein:
the aroma vapor dispenser further includes a housing, platform, or holder; and
each aroma fluid reservoir unit within the set of aroma fluid reservoir units is removably matingly engageable with the housing, platform, or holder.

24. An aroma fluid reservoir unit for an aroma vapor dispenser, comprising:
a receptacle having a cavity therein for carrying an aroma fluid volume; and
an aroma fluid vaporizing element fluidically coupled to the cavity, the aroma fluid vaporizing element electrically couplable to and activatable by way of the main control unit; and
an identification unit storing an electronically-readable reservoir unit identifiers (IDs) corresponding to the fluid reservoir units.

25. The aroma fluid reservoir unit of claim 24, wherein the electronic identification unit comprises one of a microcontroller and a radio frequency ID (RFID) tag.
